(19) United States
(12) Patent Application Publication
(10) Pub. No.: US 2025/0375164 A1
(43) Pub. Date: Dec. 11, 2025

(54) PHASE-LOCK AVERAGING FOR NOISE REMOVAL FROM PHYSIOLOGICAL SIGNALS

(71) Applicant: JRE Star Investment Holdings, LLC, Farmington, UT (US)

(72) Inventors: Tyler J. Jarvis, Provo, UT (US); Robert C. Davis, Provo, UT (US); Annika C. Knudson, Los Angeles, CA (US); Bryce A. Lunceford, Mapleton, UT (US)

(21) Appl. No.: 19/230,011

(22) Filed: Jun. 5, 2025

Related U.S. Application Data
(60) Provisional application No. 63/656,277, filed on Jun. 5, 2024.
(60) Provisional application No. 63/703,693, filed on Oct. 4, 2024.

(51) Int. Cl. A61B 5/00; A61B 5/024; A61B 5/145

(52) U.S. Cl. CPC A61B 5/7203; A61B 5/02416; A61B 5/14532; A61B 5/7235; A61B 5/7275

(57) ABSTRACT

Methods and systems for removing noise from signal data obtained from a non-invasive blood monitor. In some implementations, the method may comprise receiving signal data from a non-invasive blood monitor and detrending at least a portion of the signal data to create a detrended signal. The detrended signal may then be stacked into a matrix. A singular value decomposition of the matrix may be taken of the matrix, which may be used to estimate one or more features of the signal that may be used to reconstruct the signal in some cases.

### CROSS-REFERENCE TO RELATED APPLICATIONS

This application claims the benefit under 35 U.S.C. § 119 (e) of U.S. Provisional Patent Application No. 63/656,277, which was filed Jun. 5, 2024, and titled “Phase Lock Averaging for removing noise from almost-periodic signals with non-constant frequency and irregular shape.” This application also claims the benefit under 35 U.S.C. § 119 (e) of U.S. Provisional Patent Application No. 63/703,693, which was filed Oct. 4, 2024, and titled “Optimal Wavelength Standardization.” Both of the aforementioned applications are hereby incorporated herein by reference in their entireties.

### SUMMARY

Disclosed herein are methods, which may be incorporated into various systems, for reducing and/or removing noise from signals, such as physiological signals, which may ultimately facilitate improved tracking of various health conditions, such as monitoring glucose or other blood analytes.

Physiological signals, such as those obtained from electrocardiograms (ECG), phonocardiograms (PCG), and electroencephalograms (EEG), and others are crucial for diagnosing, monitoring, and treating various medical conditions. These signals often contain noise, posing challenges for both manual and automated interpretation. This disclosure addresses the problem of noise in physiological signals, focusing primarily on photoplethysmography (PPG) spectrometer signals used for non-invasive continuous glucose monitoring. However, as those of ordinary skill in the art will appreciate, the inventive principles disclosed herein may be used in connection with a variety of other physiological signals, or even other non-physiological signals in some cases.

Two primary, novel preprocessing methods are disclosed herein: Wavelength Standardization and Phase-Lock Averaging. Wavelength Standardization ensures compatibility between measurements from different devices, enhancing the dataset available for model training. Phase-Lock Averaging, originally developed for PPG signals, significantly reduces noise in pulsatile signals, such as heartbeat signals, and in various embodiments and implementations may outperform traditional Fourier methods, particularly in high-noise environments.

This disclosure outlines practical considerations and caveats in applying Phase-Lock Averaging to real-world data, demonstrating its efficacy and limitations. The findings suggest that, despite certain drawbacks, Phase-Lock Averaging offers a robust solution for improving the quality of physiological signal analysis, with potential applications beyond PPG signals.

This work contributes valuable preprocessing techniques for physiological signal analysis, providing a foundation for further research and application in medical diagnostics and monitoring.

The realm of physiological signals includes signals from devices such as the electrocardiogram (ECG) for electrically measuring the contractions of the heart, the phonocardiogram (PCG) for measuring the acoustic sounds of the heart (like a stethoscope), and the electroencephalogram (EEG) for measuring brain activity. Such signals even include simple signals like body temperature measurements. These signals are important for diagnosing, monitoring, and treating disease.

Like all signals, physiological signals contain noise. When the amount of noise is large enough, it can sometimes be an issue for humans who manually interpret these signals. However, noise is almost always something that needs to be considered and accounted for when designing systems that can automatically interpret physiological signals. Because of this, the field of physiological signal processing has produced many interesting mathematical problems. There have been entire books written with information about how to analyze physiological signals.

In preferred embodiments and implementations, this disclosure relates to non-invasive glucose monitoring, such as non-invasive, continuous glucose monitoring. The physiological signal that is being analyzed for this task is typically a collection of simultaneously measured photoplethysmography (PPG) signals. PPG signals measure light as it is passed through the body, and are the driving technology behind pulse oximetry, a method to non-invasively measure oxygen content in the blood.

The preferred devices used in this disclosure are wearable spectrometer devices. They may be configured to shine a broad spectrum of light into the wrist and measure the intensity of the light that is reflected back to the spectrometer. In some cases, the sensor may be configured to measure the intensity of light at 128 specific wavelengths, measuring the spectrum of light that returns to the spectrometer detector. This gives much more information than would be available if only one wavelength of light was shined into the wrist via, e.g., a laser. Some of the light may penetrate deep enough to reach the radial artery, which not only means that the device is able to directly measure the optical properties of the blood (albeit obscured by the optical properties of other tissues in the wrist), but it also means that a pulsatile signal can be seen as the heart beats. Because of this, the pulsatile information of the signal is an important property for analysis.

This disclosure will present two primary methods that were developed to help in preparing the spectrometer data for analysis. The first, called Wavelength Standardization, makes measurements from separate devices that measure different wavelengths compatible with one another so that models can be trained with measurements from both devices. The second, called Phase-Lock Averaging, is a method for removing noise from the pulsatile spectrometer signal, and while it was originally developed for PPG signals, it could potentially be applied to other pulsatile physiological signals, or even other non-physiological signals containing a pulsatile component, to significantly reduce the amount of noise present.

Although the details for analyzing such signals after noise removal has taken place are not discussed herein, they can be found in, for example, U.S. patent application Ser. No. 18/955,864 titled “METHODS AND SYSTEMS FOR PROCESSING NON-INVASIVE BLOOD MONITOR DATA,” which was filed on Nov. 21, 2024 and U.S. patent application Ser. No. 18/991,182 titled “METHODS AND SYSTEMS FOR ESTIMATING BLOOD ANALYTES FROM SPECTROMETER SIGNALS,” which was filed on Dec. 20, 2024. Both of these patent applications are hereby incorporated by reference herein in their entireties.

In an example of a method for reducing signal noise from a non-invasive blood analyte monitoring system, the method may comprise detrending a signal, such as a heartbeat or other physiological signal. The signal may then be split into individual pulses, such as individual heartbeats. In some cases, a peak-finder may be used to accomplish this splitting. The pulses, such as heartbeats, may then be resampled, such as resampled to be the same length. The pulses, such as resampled heartbeats in some cases, may then be stacked into one or more matrices.

In some cases, the matrix or matrices may then be modified. For example, in some cases, the matrix/matrices may be modified in a manner that will be recoverable, such as recoverable after taking the singular value decomposition.

The singular value decomposition of the matrix or matrices may then be computed. Using the results of the singular value decomposition, the pulse shape(s) and/or amplitude(s) may then be estimated. In some cases, a reconstruction of the signal may then be made. For example, in some cases, the trend, pulse shape(s), and/or amplitude(s) may be used to create a reconstruction of the signal.

In some cases, as an alternative to using a singular value decomposition, another low rank approximation to the matrix may be used. For example, a Nonnegative Matrix Decomposition may be used to approximate the pulse shape(s) and/or amplitude(s) directly using, for example, gradient descent and/or using any desired method for Principal Components Analysis.

In another example of a method for reducing signal noise, such as signal noise from a physiological signal, the method may comprise receiving signal data, such as physiological signal data. In some cases, such data may be received from a non-invasive blood monitor, such as a wrist monitor configured to take data from the radial artery.

A trend may be estimated in the signal data. At least a portion of the signal data may then be detrended to create a detrended signal. In some cases, resampling of the signal may be performed. The detrended signal may then be split into vectors. The detrended signal may then be stacked into a matrix. An estimate for a shape(s) and/or amplitude(s) of a pulse associated with the signal may then be made. In some cases, a singular value decomposition of the matrix may be used to estimate the shape and/or amplitude(s) of the signal.

Some implementations may comprise taking a decomposition of the matrix.

Some such implementations may further comprise modifying a stacked matrix, such as modifying the stacked matrix by means of matrix multiplication, weighting, and/or mathematical decomposition. For example, in some cases, the stacked matrix may be multiplied by the Cholesky decomposition of the inverse covariance matrix. In some cases, the Eigen decomposition of the inverse covariance matrix may be used.

In some implementations, a Cholesky decomposition may be used.

In some implementations, a non-invasive blood monitor may be used, which may be configured to detect a concentration of a blood analyte, such as glucose.

In an example of a method for removing noise from signal data, such as physiological signal data obtained from a non-invasive blood monitor, the method may comprise receiving signal data and detrending at least a portion of the signal data to create a detrended signal. The detrended signal may then be stacked into a matrix, after which a singular value decomposition of the matrix may be taken.

Some implementations may further comprise taking a Cholesky decomposition of an inverse covariance matrix derived from the matrix.

Some implementations may further comprise using the singular value decomposition to estimate a shape and/or amplitude of a pulse associated with the signal data. In some cases, both a shape and one or more amplitudes of the pulse may be estimated.

In some implementations, the step of using the singular value decomposition to estimate a shape and/or amplitude of a pulse associated with the signal data may comprise estimating the shape and the amplitude of the pulse.

Some implementations may further comprise estimating a concentration of a blood analyte, such as glucose, using physiological signal data.

In an example of a system for removing noise from signal data according to some embodiments, the system may comprise a radiation generator, such as an emitter, configured to generate electromagnetic radiation. The system may further comprise one or more sensors configured to receive reflected electromagnetic radiation from the radiation generator.

The system may further comprise various functional modules, such as software modules. For example, the system may comprise a detrending module configured to detrend a signal from data obtained from the sensor.

The system may further comprise a stacking module configured to stack a detrended signal obtained from the detrending module.

The system may further comprise a singular value decomposition module configured to take a singular value decomposition of the detrended signal.

The system may further comprise a signal reconstruction module. The signal reconstruction module may be configured to estimate one or more shapes and/or amplitudes associated with the signal. In some such cases, the signal reconstruction module may further be configured to obtain a pulse shape and/or a one or more pulse amplitudes from the singular value decomposition. In some cases, the signal reconstruction module may further be configured to combine data from various modules, such as the trend, pulse shape(s), and/or pulse amplitude(s) to create a reconstruction of the signal.

In some embodiments, the signal data may comprise physiological signal data, such as heartbeat data.

Some embodiments may further comprise a triangular decomposition module configured to compute a decomposition of an inverse covariance matrix derived from the signal data. In some such embodiments, the triangular decomposition module may be configured to take a Cholesky decomposition.

In some embodiments, the sensor may comprise a spectrometer and/or the signal may comprise a photoplethysmography signal.

In an example of a method for wavelength standardization according to some implementations and embodiments, the method may comprise performing an experiment to determine the spectral sensitivity of one or more of the photodiodes used on the sensor/device. As discussed below, in some cases, this may be found to be approximately Gaussian, but other devices may have different shapes for the spectral sensitivity.

In some cases, a spectral sensitivity shape may then be determined for each of the photodiodes of the fictional “ideal” device. Typically, however, this will only need to be done once, since this fictional device is the one that all of the others are being “standardized” to.

The spectral sensitivity of the real device may then be used to construct the transformation F from the true spectrum to the measured spectrum.

The spectral sensitivity of the fictional “ideal” device may then be used to construct the transformation G from the true spectrum to the measured spectrum.

Wach transformation may then be approximated as a Riemann sum so that they can be represented as matrices. Depending on the spectral sensitivity shapes, approximations other than a Riemann sum may be used in some cases, such as Simpson's rule or Quadrature.

The transformations may then be represented as matrices using the chosen Riemann sum/Simpson's rule/Quadrature approximations.

The pseudo-inverse of the matrix F may then be computed, after which the matrix T=F\hat{ }+G may be computed by multiplying F's pseudoinverse by G.

Finally, the detected measurements from the real device may be multiplied by the matrix T to find out what the “ideal” device would have measured,

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a model of a detector;

FIG. 2 includes three graphs illustrating wavelength standardization;

FIG. 3 is a graph providing an example of wavelength standardization applied to real spectrometer data;

FIGS. 4A and 4B are graphs illustrating the mixture of white and pink noise typically contained in a physiological signal;

FIGS. 5A and 5B are graphs illustrating shapes of a synthetic signal created by repeating a pulse shape scaled by random amplitudes;

FIG. 6A consists of graphs illustrating the power spectrum of a synthetic signal;

FIG. 6B consists of graphs comparing a synthetic signal with a band pass filter applied to the synthetic signal;

FIG. 7A is a graph illustrating the power spectrum of a synthetic signal;

FIG. 7B is a graph comparing a noisy synthetic signal with a band pass filter applied to the synthetic signal;

FIG. 8 consists of graphs providing a comparison of domain restrictions for a von Mises-Fisher distribution and a Pulse Shape Prior distribution;

FIG. 9 consists of graphs comparing the PDFs of the von Mises-Fisher distribution and Pulse Shape Prior distribution in terms of the angle θ;

FIGS. 10-27 are graphs illustrating monte carlo results for PLA applied to various signals differing in amounts and types of noise added;

FIG. 28 consists of graphs showing monte carlo samples from the distributions of pulse shapes found by PLA for different kinds and amounts of noise added;

FIG. 29 is a graph illustrating PLA applied to white noise;

FIG. 30 is a graph illustrating the estimated pulse shape provided by PLA;

FIG. 31 is a graph illustrating PLA applied to pink noise;

FIG. 32 is a graph illustrating the estimated pulse shape provided by PLA;

FIG. 33 is a graph illustrating PLA applied to the noisy synthetic signal of FIGS. 7A and 7B;

FIG. 34 is a graph illustrating PLA applied to real spectrometer data;

FIG. 35 is a flowchart illustrating a method for removing noise from a physiological signal according to some implementations; and

FIG. 36 is a block diagram illustrating an example of a system for removing noise from a physiological signal according to some embodiments.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

The first method for preparing spectrometer data for analysis that will be discussed herein is Wavelength Standardization. There are two initial problems that may be addressed before any analysis on the spectrometer data. First, the spectrometer device does not just pick up light given off from the emitter. It also picks up light from the environment, called ‘ambient light.’ To account for this ambient light, the device may be configured to take a measurement approximately every minute. During this measurement, called the ‘ambient scan,’ the device turns off the emitter for several seconds and takes continuous readings during this time. This allows for an estimate of the ambient light.

Second, due to manufacturing differences between devices, the light emitted at different wavelengths can vary. Even in a single device, the light from the emitter can change over time due to the LED aging as the device is used. To account for this variability, an experiment may be run periodically (every few days to weeks) on each device to measure the light from the emitter. During the experiment, the device may be placed in front of a highly reflective material and the light is measured at each wavelength. This measurement is called the ‘brightfield.’

If we assume that the ambient light is additive with the light from the emitter and that the brightfield is a multiplicative factor, we can use the ambient scan and brightfield to preprocess the spectrometer data by doing the following:

First, for each wavelength λ, we find the median of the measurements taken during the ambient scan and call it Xλ^{ambient}. This value is taken to be the ambient light at that wavelength until a new ambient scan is performed.

Second, from the brightfield experiments, we have a vector of values Xλ^{brightfield}, one value for each wavelength.

Third, at each point in time, we then subtract off the ambient light and normalize by the brightfield to get an estimate for the amount of light returned from the emitter, giving:

$${S}_{λ}^{preprocessed} ( t ) = \frac{{S}_{λ}^{measured} ( t ) - {X}_{λ}^{ambient}}{{X}_{λ}^{brightfield}} .$$

If all of the measurements were taken on the same device, the preprocessing steps taken above would be sufficient to analyze the data. However, being limited to a single device severely limits the amount of data that can be used to train a model. Ideally, we would like to be able to use data from multiple devices.

As mentioned above, in some cases, each device may have a large number of channels, such as 128 channels, each of which measures light at different wavelengths. However, because of the way that the spectrometer is manufactured, the wavelengths that are measured in each specific channel can vary from device to device-sometimes quite significantly. Once the device is completed, an experiment must be run to determine exactly what wavelengths are being measured in each channel. This means that the data from one device cannot be directly compared to the data from another device, as the same channels may be measuring different wavelengths of light, or vice versa. In order to compare data across devices, it is therefore necessary to standardize the measurements in some way so that the same wavelengths are being measured in the same channels on each device.

This is not a straightforward problem, but one key observation is that a particular channel does not simply measure a single wavelength of light. Instead, it measures a band of wavelengths around a central wavelength. Furthermore, these bands overlap with each other to some extent.

Experimentally, it was found that for some devices, the bands of wavelengths measured by each channel are approximately Gaussian, centered at the central wavelength and with a standard deviation of approximately 10 nm. On average, the central wavelengths of neighboring channels are separated by only about 6 nm. Therefore, for a given wavelength of light at some intensity coming to the detector, the light is picked up by several channels (along with the other wavelengths that are picked up in that band), with the largest proportion of the light being picked up by the channel whose central wavelength is closest.

To state this idea more concisely and in mathematical language, suppose that at a certain time t, there is a true spectrum of light S^{true}(λ)∈L² [0, ∞) that enters the detector (and that all of this light will be picked up by the detector). Note that we have abused notation slightly here, as S^{true}(λ) is a function of wavelength, not time. Note further that S^{true}(λ) is a function of continuous wavelength, not discrete wavelength. We can think of S^{true}(λ) as a vector in infinite-dimensional space.

Then, we define S^{measured}∈R¹²⁸ to be the vector of measurements as given by the detector. This vector can be thought of as a function of discrete wavelengths, where the domain is the set of 128 wavelengths (or however wavelengths the detector can measure). We can then define the relationship between S^{true}(λ) and S^{measured}. It was found experimentally that the band of wavelengths measured by the channel centered at ξ is approximately Gaussian, centered at ξ with a standard deviation of σ=10 nm. Thus, we can define

$${w}^{detector} ( λ ; ξ ) = \frac{1}{\sqrt{2 π} σ} {e}^{- \frac{{( λ - ξ )}^{2}}{2 {σ}^{2}}}$$

to be the detector response function centered at ξ. Then,

$${S}_{ξ}^{measured} {\int }_{- ∞}^{∞} {S}^{true} ( λ ) {w}^{detector} ( λ ; ξ ) d λ . \qquad ( 2.1 )$$

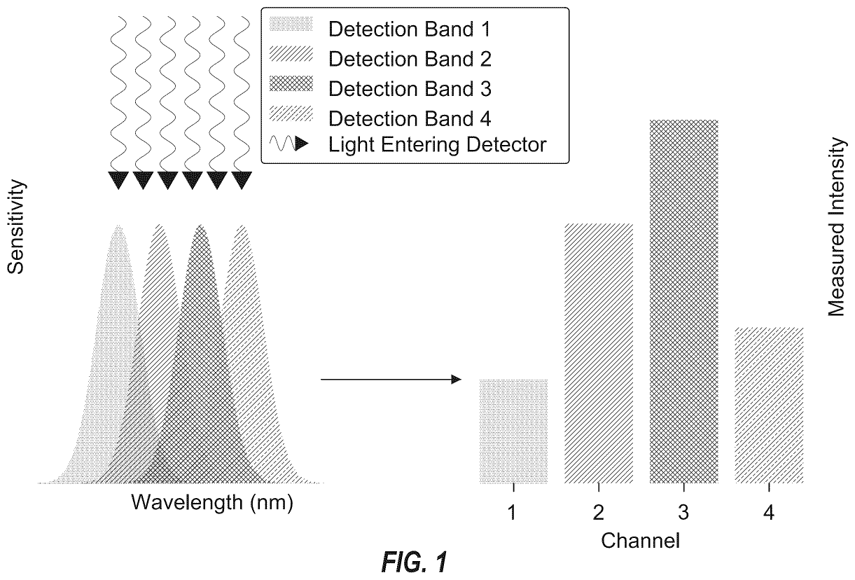

Notice that this is a linear transformation. Therefore, there is a linear operator F:L²[0, ∞)→R¹²⁸ such that S^{measured}=FS^{true}. See FIG. 1, which is an illustration of the model of the detector. The true spectrum of light S^{true}(λ) enters the detector, and the detector measures the light in bands of wavelengths centered at the central wavelengths of the channels. The light is picked up by the channels in proportion to the amount of light in the band of wavelengths measured by the channel. The detector then outputs the measurements S^{measured}.

Now, we suppose that there is some ‘ideal’ detector that we wish that all of the measurements were taken on. We can proceed in the same way as above and define S^{ideal}∈R¹²⁶ to be the vector of measurements that the ideal detector would take. The reason that the ideal detector would only take 126 measurements is that we want the wavelengths measured by all of the devices to ‘cover’ the wavelengths measured by the ideal detector, and the position of the two end channels varied between devices, some devices measuring slightly longer wavelengths and some measuring slightly shorter wavelengths. Like the real detector, there are bands of wavelengths that the ideal detector would measure, so we define w^{ideal}(λ; ξ) to be the band of wavelengths measured by the channel centered at ξ. Then, we can define the relationship between S^{true}(λ) and S^{ideal} as:

$${S}_{ξ}^{ideal} {\int }_{- ∞}^{∞} {S}^{true} ( λ ) {w}^{ideal} ( λ ; ξ ) d λ . \qquad ( 2.2 )$$

Again, this is a linear transformation, so there is a linear operator G:L²[0, ∞)→R¹²⁶ such that S^{ideal}=GS^{true}.

The goal is to answer the question: Had the ideal detector instead been the one to take the measurements, what would the measurements have looked like? Ideally, the answer would have been straightforward: Because we have that S^{measured}=FS^{true} and S^{ideal}=GS^{true}, we could have simply inverted F to get S^{ideal}=GF⁻¹S^{measured}. Unfortunately, F is not invertible (by the Fundamental Theorem of Linear Algebra), so the question is ill-posed.

Instead, we seek to find a ‘best’ answer to the question. Because F is not invertible, we will seek instead to find the Ŝ^{true} such that FŜ^{true}=S^{measured} and

$${‖ {\hat{S}}^{true} ‖}_{2}^{2}$$

is minimized.

This has the physical interpretation of being the spectrum of light with minimal power that could have given rise to the measurements taken by the detector. In other words, we want to find a function that maps

$${S}^{measured} ↦ {arg min}_{{S}^{true}} {‖ {\hat{S}}^{true} ‖}_{2}^{2} ; subj . to {FS}^{true} = {S}^{measured} .$$

This is a well-posed question, and it turns out that the answer is again a linear transformation, the Moore-Penrose pseudoinverse F⁺ of F. Once we have Ŝ^{true}, we can compute S^{ideal}=GŜ^{true}.

Thus, we can define a linear operator T: R¹²⁸→R¹²⁶ as T=GF⁺ that finds a ‘best’ estimate for the measurements that would have been taken by the ideal detector, given the measurements taken by the real detector. Notice that this linear transformation, once computed, does not actually need to work with the infinite-dimensional space L²[0, ∞) as it simply acts on the measurements taken by the detector. It can be represented as a matrix in R^{126×128}.

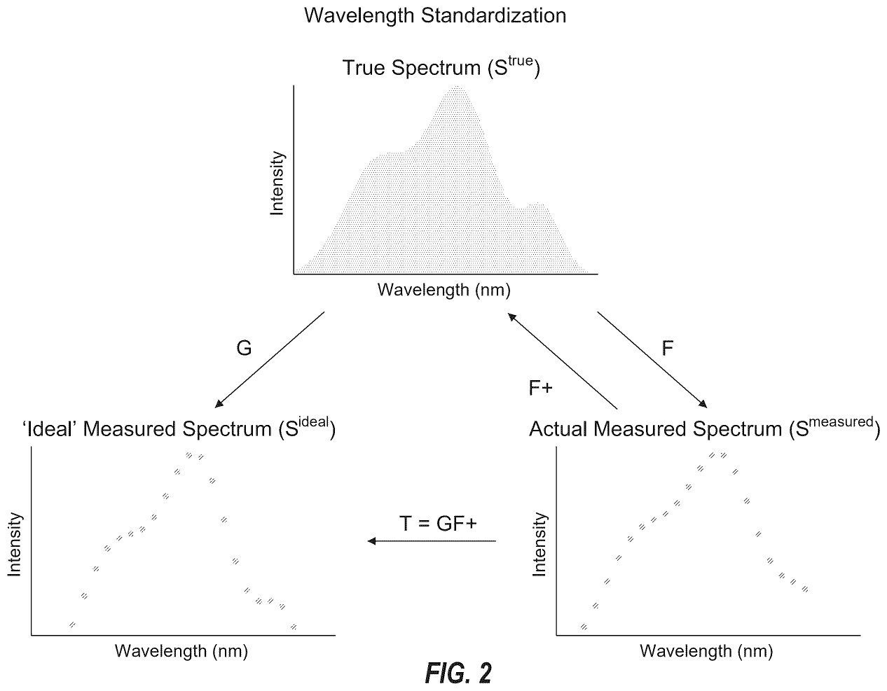

See FIG. 2, which is an illustration of wavelength standardization. A true spectrum physically exists but is not measured. Instead, the device measures the true spectrum at a discrete number of wavelength bands (see FIG. 1). Each device measures different wavelength bands, so their measurements must be standardized to a fictional ‘ideal’ device. The transformations from the true spectrum to the measured spectrums can be represented as linear transformations. To standardize the wavelengths, the true spectrum is estimated by computing the Moore-Penrose pseudoinverse of the measured spectrum. Once the estimation is made, a transformation can be made from the estimated spectrum to the ideal measured spectrum.

To speed up the computation, the standardization can be done by a single transformation from the actual measured spectrum to the ideal measured spectrum.

In practice, it may not be necessary to compute the pseudoinverse of F, where F is linear operator that acts on L²[0, ∞). This is because the integrals in equations (2.1) and (2.2) can be arbitrarily well-approximated by a Riemann sum (assuming that the functions are well-behaved). Hence the linear operators F and G can be approximated by a matrix where the rows are w^{detector}(λ; ξ) and w^{ideal}(λ; ξ), respectively, evaluated at selected points in their domains. This makes the computation of the pseudoinverse of F a simple matrix operation. The matrix T is then found by multiplying the matrix G by the pseudoinverse of the matrix F. We found that using 350 points was sufficient to get a good approximation for T. Above that point, there was little change in the matrix T.

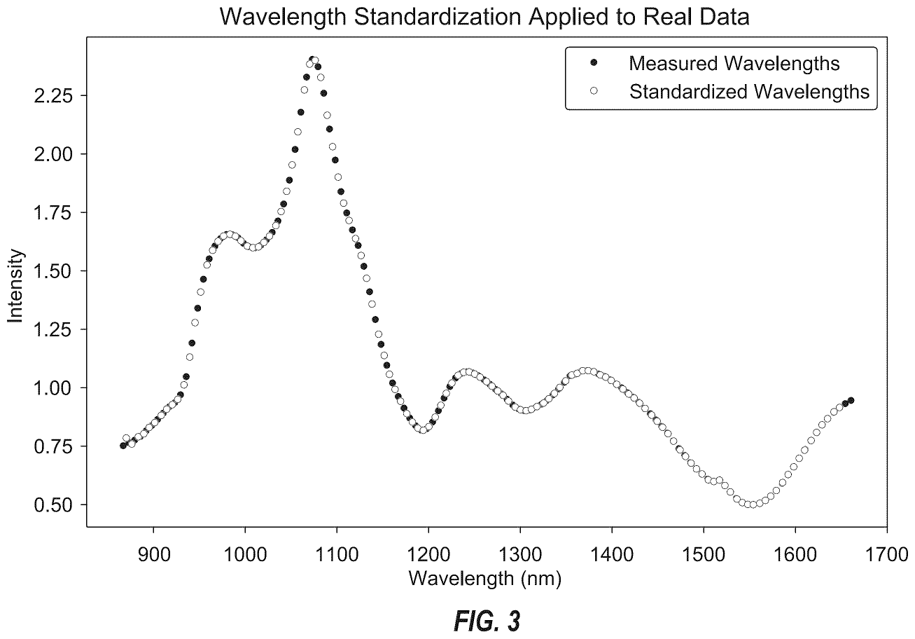

FIG. 3 illustrates an example of wavelength standardization applied to real spectrometer data. The measured spectrum from the detector is shown along with the estimated spectrum that would have been measured by the ideal detector. The estimated spectrum falls where one would expect it to be between the measured spectrum.

The phase-lock averaging aspects and features of this disclosure will now be described in greater detail.

Physiological signals, including (and especially) PPG signals are very noisy. There are many ways that noise can be introduced into physiological signals. In addition to random noise produced by electronic noise, shot noise from photons, and noisy physiological processes, there are additional challenges, such as noise from electricity mains, powerline drift, motion artifacts, and baseline drift. Because of this, there is typically a lot of preprocessing that must be done to make a PPG signal usable.

While acknowledging that other sources of noise exist and are significant challenges to overcome, in this disclosure, we will only focus primarily on random noise caused by electronics, photons, or fast, unpredictable physiological processes. The other forms of noise are more difficult to analyze mathematically as they are not described by familiar random processes. Often, there is no good way to deal with these kinds of noise, and the corrupted signal must simply be removed.

Random noise is difficult to work with as well, and many different methods have been analyzed to deal with or remove it. These methods include simple filters like moving average filters, convolution-based filters, and cross-correlation-based filters; and more complicated filters like Butterworth, Chebyshev, elliptic, and Weiner filters. One recent paper, published in 2018, found the fourth-order Chebyshev Type II filter to be particularly suited to PPG signals. Not all of these filters have an immediate, direct reliance on the Fourier transform, but because convolution and cross-correlation in the time domain are equivalent to multiplication in the Fourier domain, these methods will be referred to herein as ‘Fourier Methods.’

It should be noted that there have been many attempts at removing noise from PPG signals. Not all of the attempts fall under the category of Fourier Methods. For example, one such attempt uses wavelets to remove noise from PPG signals. Others use the Kalman filter to remove noise.

Regardless, Fourier methods seem to be the most widely utilized kinds of filters in general use. Therefore, in this disclosure, comparisons will be made between Phase-Lock Averaging and Fourier methods.

Fourier Methods are a tried and true way to remove noise from a wide variety of signals. They have found use in diverse applications such as audio processing, image processing, compression, communications, and more. Their nearly universal adoption is due, among other things, to their simplicity, flexibility, and speed. A filter can be designed to meet many constraints about the shape of the frequency response, the filter delay, and computational resources available. Because of the FFT, filters can be made to be nearly instant, even with low computational power.

Despite the many advantages and versatility of Fourier methods, however, they have a few properties that make them less than ideal for physiological signals. We should note that the end goal when processing physiological signals is usually much different than the end goal for other applications. In physiological signal processing, the goal is often to recover, as closely as possible, the exact shape of the signal so that downstream tasks can properly analyze it. In contrast, in audio and image processing the goal is rarely to produce a filtered signal that is exactly the same as the source signal. Instead, the goal is to create a filtered signal that sounds clear enough for the human ear to understand, or to produce an image that is clean enough for a human eye to see a clear picture, or to add a special effect that sounds interesting, etc. It does not matter if the filter introduces some artifacts into the signal, because it was never the goal to recreate the signal exactly.

In compression applications, (which some mistake for denoising algorithms) the goal is to recreate the original signal as exactly as possible, but not with the goal of specifically targeting noise for removal. Fourier-based compression algorithms, in a nutshell, transform the signal to an alternate basis, and then remove as many of the basis elements as possible while keeping the signal within a level of tolerance. In some applications, it happens that the noise is so small compared to the signal of interest, that compression removes the noise.

However, for physiological signals, the noise is typically large enough that, from the perspective of the compression algorithm, the signal and noise are indistinguishable. The result is that both signal and noise get removed, so artifacts appear in the filtered signal.

In communications signals applications, the goal is to remove noise and recover the original signal, the same goal as that in physiological signals applications. However, there is one crucial difference. Communications engineers have control over the form of the source signal and, therefore, are able to more easily distinguish between what constitutes signal and what constitutes noise. Very aggressive filters can be designed because the goal is to pass information, and to a certain extent the engineers know what form the information will take.

In physiological signals, on the other hand, there is no control over the source signal. Often, it is not even known ahead of time what exactly a signal will look like, and the goal is to find out what form the source signal takes.

The goal of filtering in physiological signals, though at first glance similar to other applications, is therefore rather different than the goals of other signal processing problems. Concisely stated, the goal of physiological signal processing is to distinguish, as clearly as possible, the source signal from the noise, and the form of the source signal is not entirely known.

Fourier methods are not particularly well-suited to the goal of physiological signal processing, as discussed below. However, by making some assumptions about the form of the source signal (that it is pulsatile and that we can find the beginning and end of the pulses), we can design a filter that is better suited to the goal of physiological signal processing.

A common misconception is that ‘signal’ is contained in lower frequencies, and that ‘noise’ is contained in higher frequencies. There is some truth to this, which is the reason that Fourier methods work at all. Usually, the signal of interest is mostly contained in the lower frequencies. Noise, by contrast, is usually contained all throughout the frequency spectrum. This is a little bit counterintuitive, because noise looks like it is a very high frequency signal—but a quick way to see that it is actually contained all throughout the spectrum is to consider simple Gaussian white noise centered at 0 with a covariance matrix of I.

Such a distribution is symmetric in every direction, meaning that it will look the same in any orthonormal basis. Then, considering the fact that the Fourier transform is just an orthonormal change of basis, the distribution would look unchanged: contained in all frequencies. With that said, what allows Fourier filters to work is that if the power of the signal is strong enough in lower frequencies relative to the noise, then noise will not affect the signal too much in those frequencies. Then, since almost all of the power of the signal is contained in lower frequencies while the power of the noise is contained all throughout the spectrum, filtering out the higher frequencies reduces the total power of the noise.

Because noise is contained all throughout the frequency spectrum, filters never remove all noise. There will always be at least some noise that is allowed through the filter. Issues can arise when the signal is not significantly larger than the noise in the frequencies that pass through the filter. When that happens, noise is passed through the filter—but it does not always look like noise.

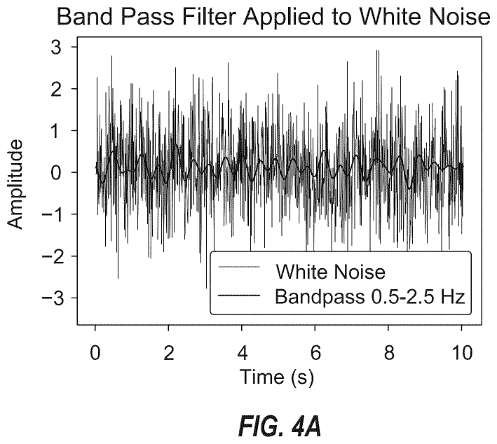

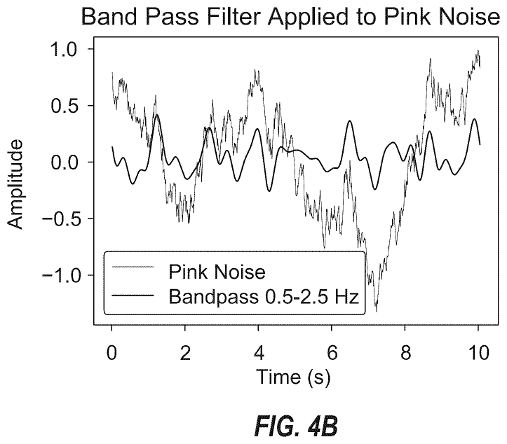

We are used to noise looking erratic and unpredictable. However, when noise is passed through a filter, the result can be a smooth, oscillatory signal (see FIGS. 4A and 4B). As demonstrated in FIGS. 4A and 4B, physiological signals, including signals from PPG devices, typically contain a mixture of white and pink noise. To show the effect of applying a filter to noise, white noise and pink noise were generated and then a band pass filter was applied to the resulting signal. A human heart beats in the range of 0.5 Hz to 2.5 Hz, so the pass band was specifically chosen to include this range. In both plots in these figures, what appears to be a signal can be seen. This is especially true of the pink noise plot, where what looks like a repeating wave can be seen fairly clearly. These plots show that a filter can easily produce a false signal from pure noise.

Unlike white noise, which has a uniform power spectrum over all frequencies, pink noise has a power spectrum that is proportional to 1/A, meaning that it is especially strong at low frequencies. The result is that, unless one is aware of how noise interacts with the filter, they could easily think there is a signal where there is none.

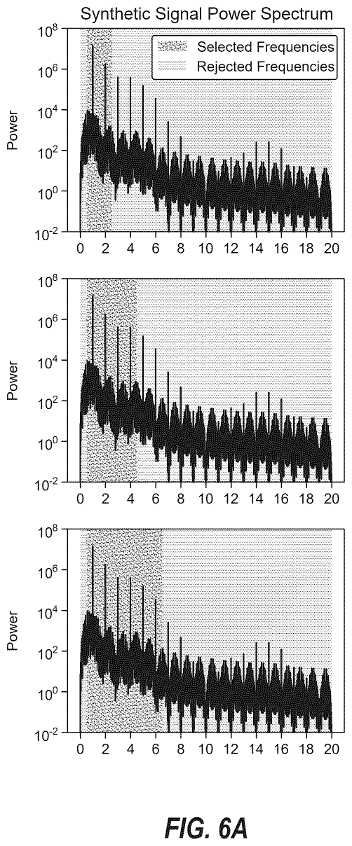

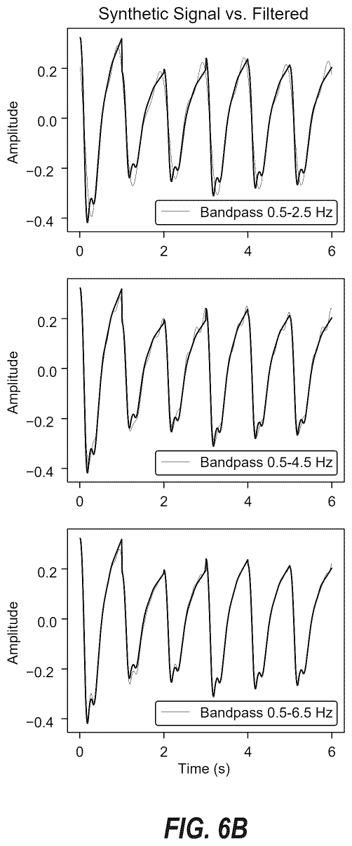

A poorly designed filter can introduce artifacts into the signal. One of the significant challenges to designing a filter is deciding which frequencies to allow to pass through and which to attenuate. For physiological signals where the underlying signal is related to the pulsing of the heart, for example, or another physiological process, one might think that the band from 0.5 Hz to 2.5 Hz would be sufficient to capture the signal, since nearly all human adults at rest will have a heart rate that falls comfortably in this band. This is not an adequate pass band, though, as can be seen in FIGS. 6A and 6B.

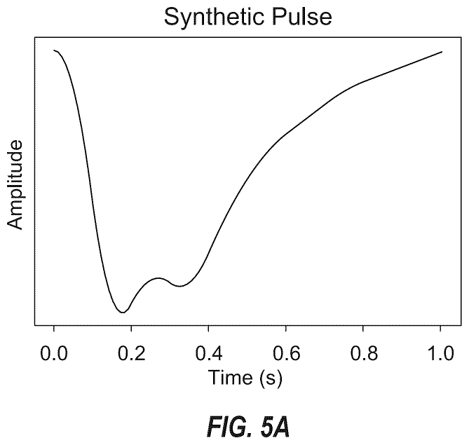

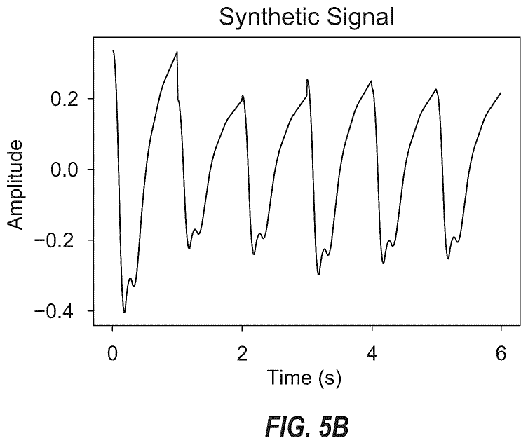

The reason is that, like musical instruments, physiological signals have ‘overtones’ or ‘harmonics.’ These are frequencies other than the main frequency that contain significant amounts of the power of the signal. Failing to also allow these frequencies to pass through the filter results in artifacts in the filtered signal, as shown in FIGS. 5A and 5B.

As shown in these figures, a synthetic signal was created by repeating a pulse shape scaled by random amplitudes 300 times to look like a typical PPG signal. The pulse shape was designed to mimic a typical pulse seen from a PPG. It is one second in length, which is approximately the length of a human heartbeat at rest. Each pulse was given a random amplitude, sampled from a normal distribution centered at two with a standard deviation of 0.4. The choice of amplitudes was somewhat arbitrary, but the signal looks (up to scaling) very similar to what a PPG device would measure. Notice that there are some artifacts in the synthetic signal at the points where the pulse repeats. This is because the pulse shape is centered at 0 to match the detrending method that will be used, as discussed below. One way that these artifacts could potentially be removed is to use a detrending method that places the start end of pulses at 0 instead of centering the pulses.

To create FIGS. 6A and 6B, a synthetic signal was generated to approximate the signal that would be seen by a PPG device monitoring a heart beating at 1 Hz. To do this, a synthetic pulse was created, which looks similar to a heartbeat. As mentioned above, this synthetic pulse was repeated 300 times, then each pulse was multiplied by a random amplitude drawn from a normal distribution centered at two with a standard deviation of 0.4. The result can be seen in FIGS. 5A and 5B.

While designing a band pass filter, it is important to allow enough of the signal to pass so that it can be sufficiently well-recovered. The plots of FIGS. 6A and 6B show the effect of a band pass filter on a signal without noise. Notice that there are large spikes at the integer values on the power spectrum plots. These spikes contain most of the power of the signal, so enough of them must be allowed to pass to adequately recover the signal. If the pass band is too small, the shape of the pulses is not correct. Notice that for the 0.5-4.5 Hz band pass filter, ripples appear. These ripples are artifacts of the band pass filter, but might be confused for true features of the pulse. Using the 0.5-6.5 Hz band pass filter, the signal is adequately recovered.

Once the synthetic signal was created, a band pass filter was applied to the synthetic signal. To apply the band pass filter, the FFT was taken of the synthetic signal, and frequencies outside the pass band were multiplied by zero before performing the inverse FFT. This gives as close an approximation as possible to the unattainable ‘ideal’ filter. In practice, this kind of filtering is rarely done because it cannot be done in real-time without introducing ringing artifacts. Real-time filtering requires the use of a windowing function in combination with carefully designed convolution kernels. This is not addressed herein for simplicity. However, because convolution in the time domain is the same as multiplication in the Fourier domain, the same principles as have been discussed throughout this section apply. The additional considerations that need to be taken to make real-time filtering work can introduce additional artifacts, including phase-shifts—even when done properly. Because of this, FIGS. 6A and 6B represent the best possible case: A periodic signal in a setting where the amount of time required to filter the signal does not matter. Real-time filtering would make the artifacts slightly worse.

Notice that in the power spectrum of the synthetic signal shown in FIGS. 6A and 6B, there are large spikes at each integer frequency value. These contain a significant amount of the power of the signal. It is not until the band pass filter is fairly wide at 0.5 Hz to 6.5 Hz that the filtered signal resembles the original signal. Notice also that when the filter width is too small, like for the 0.5 Hz to 4.5 Hz band, ripples appear in the decay phase of the heartbeat while the signal ‘ramps up’ for the fast, quick power-stroke. The mechanism that causes this is the same as for Gibbs' phenomenon. These artifacts can easily be confused for real features of the pulse. Finally, because every person's heart beats slightly differently, this analysis in no way suggests that 0.5 Hz to 6.5 Hz will always be wide enough to capture the signal. Some signals may need an even wider pass band.

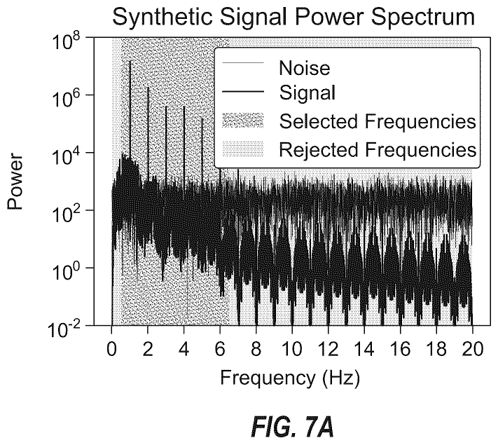

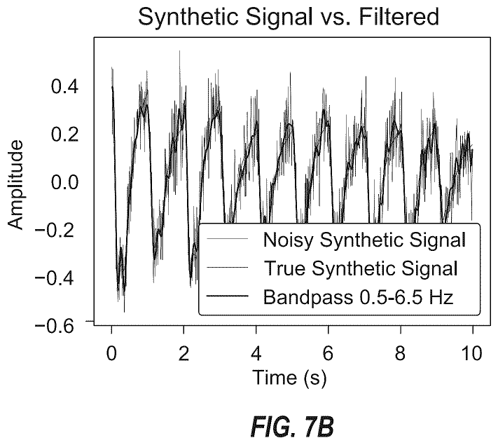

As described above, in order to adequately reconstruct the signal, the filter must have a wide enough pass band. Otherwise, artifacts are introduced. Unfortunately, the signal considered in the previous subsection did not contain any noise. What happens when noise is included? Because the pass band is so large, a lot of noise makes it through the filter (See FIGS. 7A and 7B).

As shown in these figures, The time delay that the filter was permitted to take for this figure was 300 seconds. In order to adequately recover the shape of the signal, a wider pass band must be used. However, the tradeoff to using a wider pass band is that more noise gets passed through the signal. In FIG. 7A, the power contained in the noise can be seen compared to the power contained in the signal. On the plot of FIG. 7B, the result of adding the noise to the signal (noisy synthetic signal) can be compared to the true signal and the estimated signal from the band pass filter. Notice that the band pass filtered signal still contains a substantial amount of noise, which cannot be eliminated without shrinking the pass band.

Using a thinner pass band introduces artifacts into the reconstructed signal. Compare FIGS. 6A and 6B to FIGS. 7A and 7B. In addition, by the same argument at the beginning of this section, the noise that makes it through the filter does not always look like noise. In FIGS. 7A and 7B, some of the spikes that show up in the filtered signal appear in the same place and roughly the same amplitude on each of the pulses, and could therefore be confused for actual features of the pulse.

This is one of the most significant issues in removing noise from physiological signals. In order to not introduce artifacts in the signal from the filter, the pass band must be large. But making the pass band large fails to remove enough of the noise. The goal of physiological signal processing is a tall order. However, throughout this paper, we will see that by making a few assumptions about the form of the signal and the form of the noise, we gain some leverage that will allow us to remove a significant amount of the noise while also avoiding introducing filtering artifacts into the pulse shape.

The Phase-Lock Averaging and its assumptions will now be discussed in greater detail. In an exemplary application, light shines from an emitter, goes through the skin of a user, and is detected by the photodetector. We can model the raw signal returned from the detector as

$${S}^{raw} ( t ) = a ( t ) S ( t ) + b ( t ) + ε ( t ) ,$$

where S(t) is the ‘pulse shape’ of the signal, a(t) is the amplitude, b(t) is the trend or ‘baseline,’ and ε(t) is a stochastic process representing noise. We assume that the amplitude and baseline are slowly varying functions of time and that the noise is a stationary Gaussian process with mean zero and known covariance. We also assume that the pulse shape is a periodic function of time with period T and is centered at zero. In real physiological signals, the pulse shape is not exactly periodic as there can be some variation in heart rate. We address this issue in greater detail below.

In preferred implementations and related embodiments, before performing any estimation, the trend is estimated and removed from the raw signal. This is referred to herein as “detrending” and can be done in a variety of ways. The method that employed herein was to take a moving average over a window of length T to estimate the trend.

This makes use of the fact that S(t) is almost periodic with period T and that a(t) and b(t) move slowly. Taking the expectation, we can define an estimate for the trend as:

$$\begin{matrix}\hat{b} ( τ ) = 𝔼 [ {\sum }_{t = τ - T / 2}^{τ + T / 2} {S}^{raw} ( t ) ] \\ = 𝔼 [ {\sum }_{t = τ - T / 2}^{τ + T / 2} a ( t ) S ( t ) + b ( t ) + ε ( t ) ] \\ = \frac{1}{T} [ {\sum }_{t = τ - T / 2}^{τ + T / 2} a ( t ) S ( t ) + b ( t ) + 𝔼 [ ε ( t ) ] \\ = \frac{1}{T} [ {\sum }_{t = τ - T / 2}^{τ + T / 2} a ( t ) S ( t ) + b ( t ) \\ \approx \frac{1}{T} {\sum }_{t = τ - T / 2}^{τ + T / 2} b ( t ) \\ \approx b ( τ ) .\end{matrix}$$

The first approximation comes from the fact that the pulse shape is periodic (except for varying amplitude) and the second approximation comes from the fact that b(t) is slowly varying. This gives an estimate of the trend at each time step. The trend can then simply be removed from the raw signal by subtracting this estimate from the raw signal, giving:

$$\begin{matrix}{S}^{detrend} ( t ) = {S}^{raw} ( t ) - \hat{b} ( t ) \\ \approx a ( t ) S ( t ) + ε ( t ) .\end{matrix}$$

It should be noted that while S^{detrend} is technically not actually equal to a(t)S(t)+ε(t), it is a good approximation, and for the remainder of the paper we will treat it as if S^{detrend}=a(t)S(t)+ε(t). This assumption is required for the proof of the optimality of the Phase-Lock Averaging estimator. In other words, PLA is only as optimal as the detrending method is accurate, which admittedly can be difficult in certain cases.

Some implementations and related embodiments may comprise discretization. Suppose that S(t) is a periodic function of time with period T and that a(t) is a step function of T-length intervals. Then, S(t)=S(t+T) for all t and t+T in its domain, and we can write a(t) as:

$$a ( t ) = {{\sum }_{k = 1}}^{K} {a}_{k} {1}_{[ ( k - 1 ) T , kT )} ( t ) .$$

Assuming that measurements are taken at discrete points in time, we can instead think of S(t) as a vector of length T. We write Sᵢ=S(tᵢ), where tᵢ is the i-th time point. This allows us to rewrite the detrended signal as K random vectors:

$${S}_{k}^{detrend} = {a}_{k} S + {ε}_{k}$$

with εₖ˜N (0, Σ) a multivariate normal with mean zero and covariance Σ.

To motivate the reasoning behind the PLA estimator, we first will consider the model of the signal described above in two cases: 1. known pulse shape and 2. known amplitudes. We will begin with the simpler case: that of known pulse shape.

We can write the likelihood L(a) as follows:

$$\begin{matrix}L ( a ) = P ( {S}_{1}^{detrend} , … , {S}_{K}^{detrend} \vert a , S ) \\ = {\prod }_{k = 1}^{K} \frac{1}{\sqrt{{( 2 π )}^{T} det ( \sum )}} exp ( - \frac{1}{2} {( {S}_{k}^{detrend} - {a}_{k} S )}^{T} {\sum }^{- 1} ( {S}_{k}^{detrend} - {a}_{k} S ) ) .\end{matrix}$$

To find an optimal estimator for a, we must maximize this likelihood. To do so, we can instead maximize the logarithm of the likelihood since the logarithm is a monotone increasing function. Taking the log of the likelihood gives:

$$\begin{matrix}ℓ ( a ) = log ( {\prod }_{k = 1}^{K} \frac{1}{\sqrt{{( 2 π )}^{T} det ( \sum )}} exp ( - \frac{1}{2} {( {S}_{k}^{detrend} - {a}_{k} S )}^{T} {\sum }^{- 1} ( {S}_{k}^{detrend} - {a}_{k} S ) ) \\ = {\prod }_{k = 1}^{K} log \frac{1}{\sqrt{{( 2 π )}^{T} det ( \sum )}} exp ( - \frac{1}{2} {( {S}_{k}^{detrend} - {a}_{k} S )}^{T} {\sum }^{- 1} ( {S}_{k}^{detrend} - {a}_{k} S ) ) \\ = {\prod }_{k = 1}^{K} ( log ( \frac{1}{\sqrt{{( 2 π )}^{T} det ( \sum )}} ) - \frac{1}{2} {( {S}_{k}^{detrend} - {a}_{k} S )}^{T} {\sum }^{- 1} ( {S}_{k}^{detrend} - {a}_{k} S ) ) \\ = K log ( \frac{1}{\sqrt{{( 2 π )}^{T} det ( \sum )}} ) - \frac{1}{2} {\prod }_{k = 1}^{K} {( {S}_{k}^{detrend} - {a}_{k} S )}^{T} {\sum }^{- 1} ( {S}_{k}^{detrend} - {a}_{k} S ) ) .\end{matrix}$$

To maximize this function, we take the derivative of [symbol] (a) with respect to each aₖ and set it equal to 0. We find that:

$$0 = - {S}^{T} {\sum }^{- 1} ( {S}_{k}^{detrend} - {a}_{k} S )$$

$$0 = - {S}^{T} {\sum }^{- 1} {S}_{k}^{detrend} + {a}_{k} {S}^{T} {\sum }^{- 1} S$$

$$- {a}_{k} {S}^{T} {\sum }^{- 1} S = - {S}^{T} {\sum }^{- 1} {S}_{k}^{detrend}$$

$${a}_{k} = \frac{{S}^{T} {\sum }^{- 1} {S}_{k}^{detrend}}{{S}^{T} {\sum }^{- 1} S} .$$

Therefore, the optimal estimate for aₖ is:

$$\hat{a} ( k ) = \frac{{S}^{T} {\sum }^{- 1} {S}_{k}^{detrend}}{{S}^{T} {\sum }^{- 1} S} .$$

Although not strictly required for the problem to be solvable, we usually restrict the allowable choices of pulse shape to be such that ∥S∥₂=1 to separate information about the pulse shape and information about the amplitude. Therefore, if Σ is a multiple of the identity matrix, this estimate can be nicely thought of as simply finding the cross-correlation between S and Sₖ^{detrend}.

The case of known amplitudes is similar. As before, we can start with the likelihood (except this time S is allowed to vary), and we arrive at the same expression for the log-likelihood:

$$ℓ ( S ) = K log ( \frac{1}{\sqrt{{( 2 π )}^{T} det ( \sum )}} ) - \frac{1}{2} {\sum }_{k = 1}^{K} {( {S}_{k}^{detrend} - {a}_{k} S )}^{T} {\sum }^{- 1} ( {S}_{k}^{detrend} - {a}_{k} S ) .$$

Taking the derivative of [symbol] (S) with respect to S gives:

$$0 = {\sum }_{k = 1}^{K} {a}_{k} {\sum }^{- 1} ( {S}_{k}^{detrend} - {a}_{k} S )$$

$${\sum }_{k = 1}^{K} {a}_{k}^{2} {\sum }^{- 1} S = {\sum }_{k = 1}^{K} {a}_{k} {\sum }^{- 1} {S}_{k}^{detrend}$$

$${\sum }^{- 1} S {\sum }_{k = 1}^{K} {a}_{k}^{2} = {\sum }^{- 1} {\sum }_{k = 1}^{K} {a}_{k} {S}_{k}^{detrend}$$

$$S = \frac{{\sum }_{k = 1}^{K} {a}_{k} {S}_{k}^{detrend}}{{\sum }_{k = 1}^{K} {a}_{k}^{2}} .$$

Therefore, the optimal estimate for S is:

$$\hat{S} = \frac{{\sum }_{k = 1}^{K} {a}_{k} {S}_{k}^{detrend}}{{\sum }_{k = 1}^{K} {a}_{k}^{2}} .$$

This can be thought of as a kind of weighted average over the observations.

Notice that we did not require ∥S∥₂=1 like we did for the case of known pulse shape. This was deliberate. If we were to enforce this condition and perform the optimization via Lagrange multipliers, the resulting optimization problem becomes solvable only numerically.

In addition, it is not a very natural choice to constrain the pulse shape, since it is the amplitudes and the data that determine the optimal pulse shape. In the model of the signal given above (Sₖ^{detrend}=aₖS+εₖ) if the amplitudes are fixed and the pulse shape is constrained, then that constrains Ek, the noise term! This is not ideal since the noise terms should be drawn from a Gaussian distribution.

Instead, if we want to ensure that ∥Ŝ∥₂=1, we should restrict the allowable choices for the amplitudes to values which result in pulse shapes that have norm 1. This mirrors what was done in the case of known pulse shape. There, it was stated that the allowable values for pulse shape are usually restricted to having norm one.

Considering this idea, what is the restriction that should be put on the amplitudes? Assuming that ∥Ŝ∥₂=1 in the equation for Ŝ we can multiply both sides by their transpose to get:

$${\hat{S}}^{T} \hat{S} = {( \frac{{\sum }_{k = 1}^{K} {a}_{k} {S}_{k}^{detrend}}{{\sum }_{k = 1}^{K} {a}_{k}^{2}} )}^{T} ( \frac{{\sum }_{k = 1}^{K} {a}_{k} {S}_{k}^{detrend}}{{\sum }_{k = 1}^{K} {a}_{k}^{2}} )$$

$$1 = {( \frac{{\sum }_{k = 1}^{K} {a}_{k} {S}_{k}^{detrend}}{{\sum }_{k = 1}^{K} {a}_{k}^{2}} )}^{T} ( \frac{{\sum }_{k = 1}^{K} {a}_{k} {S}_{k}^{detrend}}{{\sum }_{k = 1}^{K} {a}_{k}^{2}} )$$

$${( {\sum }_{k = 1}^{K} {a}_{k}^{2} )}^{2} = {( {\sum }_{k = 1}^{K} {a}_{k} {S}_{k}^{detrend} )}^{T} ( {\sum }_{k = 1}^{K} {a}_{k} {S}_{k}^{detrend} ) .$$

This is the restriction that must be satisfied in order to ensure that ∥Ŝ∥₂=1. Admittedly, this restriction is difficult to satisfy since it involves finding the roots of a quartic polynomial in R^{K}.

Despite that, one reasonable way to ensure that the restriction on the amplitudes is satisfied is to assume that only relative amplitudes are known. E.g., it is known that one pulse has twice the amplitude of another, but the exact value of the amplitude is not known. Doing this, we assume that for each k, we know qₖ, where each aₖ=rqₖ for some (unknown) r. Substituting this into the equation for Ŝ we get:

$$\hat{S} = \frac{{\sum }_{k = 1}^{K} {rq}_{k} {S}_{k}^{detrend}}{{\sum }_{k = 1}^{K} {r}^{2} {q}_{k}^{2}}$$

$$\hat{S} = \frac{r {\sum }_{k = 1}^{K} {q}_{k} {S}_{k}^{detrend}}{{r}^{2} {\sum }_{k = 1}^{K} {q}_{k}^{2}}$$

$$\hat{S} = \frac{1}{r} \frac{{\sum }_{k = 1}^{K} {q}_{k} {S}_{k}^{detrend}}{{\sum }_{k = 1}^{K} {q}_{k}^{2}}$$

$$r \hat{S} = \frac{{\sum }_{k = 1}^{K} {q}_{k} {S}_{k}^{detrend}}{{\sum }_{k = 1}^{K} {q}_{k}^{2}} .$$

Then, it is easy to choose r so that ∥S∥₂=1. Multiplying each side of the equation by its transpose, we arrive at:

$${( r \hat{S} )}^{T} ( r \hat{S} ) = {( \frac{{\sum }_{k = 1}^{K} {q}_{k} {S}_{k}^{detrend}}{{\sum }_{k = 1}^{K} {q}_{k}^{2}} )}^{T} ( \frac{{\sum }_{k = 1}^{K} {q}_{k} {S}_{k}^{detrend}}{{\sum }_{k = 1}^{K} {q}_{k}^{2}} )$$

$${r}^{2} = {( \frac{{\sum }_{k = 1}^{K} {q}_{k} {S}_{k}^{detrend}}{{\sum }_{k = 1}^{K} {q}_{k}^{2}} )}^{T} ( \frac{{\sum }_{k = 1}^{K} {q}_{k} {S}_{k}^{detrend}}{{\sum }_{k = 1}^{K} {q}_{k}^{2}} )$$

$$r = \pm \sqrt{{\frac{{\sum }_{k = 1}^{K} {q}_{k} {S}_{k}^{detrend}}{{\sum }_{k = 1}^{K} {q}_{k}^{2}} )}^{T} ( \frac{{\sum }_{k = 1}^{K} {q}_{k} {S}_{k}^{detrend}}{{\sum }_{k = 1}^{K} {q}_{k}^{2}} ) .}$$

It is clear from context which choice of r should be used (if it makes sense for certain amplitudes to be negative).

At this point, one might think that we could simply combine the above two methods to get an estimate for both the pulse shape and the amplitude. However, there are a few issues with this. First, unlike the case of known amplitude, we must constrain the pulse shape to have norm 1. This is because the pulse shape and amplitudes are multiplied together, so the pulse shape could be scaled arbitrarily while inversely scaling the amplitudes. As mentioned above, this leads to a problem which is solvable only numerically. Second, doing this gives a nonlinear system of equations that must be solved simultaneously. The pulse shape depends on the amplitudes and the amplitudes depend on the pulse shape. This makes the problem difficult to solve.

Instead, we stack the vectors Sₖ^{detrend} into a K×T matrix S^{detrend} by row, multiply it by the Cholesky decomposition of the inverse covariance matrix, and take its singular value decomposition to get an estimate for the pulse shape and amplitudes. If we write S S^{detrend} L=UΣV^{T}, where Σ⁻¹=LL^{T}, then the pulse shape is estimated as:

$$\hat{S} = \frac{{L}^{- T} {v}_{1}}{{‖ {L}^{- T} {v}_{1} ‖}_{2}}$$

Where v₁ is the first right singular vector, and the amplitudes are estimated as â=∥v₁^{T}L⁻¹∥₂σ₁u₁. Pseudocode for the full algorithm is given in “Algorithm 1” presented below.

Stated generally, Algorithm 1 comprises the following steps, which may, but need not always, be performed in the precise sequence listed below.

First, the signal, such as a heartbeat or other physiological signal, may be detrended. The signal may also be split into vectors, such as split into K period-length vectors

$${\{ {S}_{k}^{detrend} \}}_{k = 1}^{K} .$$

Each vector may, in some cases, correspond with a single pulse, such as, in the case of heartbeats, a single heartbeat.

The detrended signal and/or the vectors may then be stacked into a matrix, such as S^{detrend}. In some cases, the vectors may be resampled to be the same, or at least substantially the same, length.

A decomposition, such as a Cholesky Decomposition, may be computed. For example, in some cases, the Cholesky Decomposition of Σ⁻¹=LL^{T} may be computed.

A singular value decomposition may be taken. For example, the singular value decomposition of

$${S}^{detrend} L = {\sum }_{i = 1}^{min \{ K , T \}} {u}_{i} {σ}_{i} {v}_{i}^{T}$$

may be taken.

The pulse shape may then be estimated. For example, the pulse shape may be estimated as

$$\hat{S} = \frac{{( {L}^{- 1} )}^{T} {v}_{1}}{{‖ {( {L}^{- 1} )}^{T} {v}_{1} ‖}_{2}} .$$

The amplitudes may then be estimated. For example, one or more amplitudes may be estimated as

$$\hat{a} = {‖ {v}_{1}^{T} {L}^{- 1} ‖}_{2} {σ}_{1} {u}_{1} .$$

Theorem 3.1: The Phase-Lock Averaging estimator (Algorithm 1, for example) is the maximum likelihood estimator for the pulse shape and amplitude of a signal that can be written as

$${S}_{k}^{detrend} = {a}_{k} S + {ε}_{k}$$

where εₖ˜N (0, Σ) are i.i.d.

The likelihood of S and a given the data is:

$$L ( S , a ) = P ( {S}_{1}^{detrend} , … , {S}_{K}^{detrend} \vert S , a )$$

$$= {\prod }_{k = 1}^{K} \frac{1}{\sqrt{{( 2 π )}^{T} det ( \sum )}} exp ( - \frac{1}{2} {( {S}_{k}^{detrend} - {a}_{k} S )}^{T} {\sum }^{- 1} ( {S}_{k}^{detrend} - {a}_{k} S ) ) .$$

We desire to find the maximizer of this function. Because the logarithm is a monotone increasing function, we can maximize the log of the likelihood instead to convert the product into a sum.

$$\begin{matrix}ℓ ( S , a ) = log ( {\prod }_{k = 1}^{K} \frac{1}{\sqrt{{( 2 π )}^{T} det ( \sum )}} exp ( - \frac{1}{2} {( {S}_{k}^{detrend} - {a}_{k} S )}^{T} {\sum }^{- 1} ( {S}_{k}^{detrend} - {a}_{k} S ) ) ) \\ = {\sum }_{k = 1}^{K} log ( \frac{1}{\sqrt{{( 2 π )}^{T} det ( \sum )}} exp ( - \frac{1}{2} {( {S}_{k}^{detrend} - {a}_{k} S )}^{T} {\sum }^{- 1} ( {S}_{k}^{detrend} - {a}_{k} S ) ) ) \\ = {\sum }_{k = 1}^{K} ( log ( \frac{1}{\sqrt{{( 2 π )}^{T} det ( \sum )}} ) - \frac{1}{2} {( {S}_{k}^{detrend} - {a}_{k} S )}^{T} {\sum }^{- 1} ( {S}_{k}^{detrend} - {a}_{k} S ) ) \\ = K log ( \frac{1}{\sqrt{{( 2 π )}^{T} det ( \sum )}} ) - \frac{1}{2} {\sum }_{k = 1}^{K} {( {S}_{k}^{detrend} - {a}_{k} S )}^{T} {\sum }^{- 1} ( {S}_{k}^{detrend} - {a}_{k} S ) .\end{matrix}$$

$$K log ( \frac{1}{\sqrt{{( 2 π )}^{T} det ( \sum )}} )$$

Note that the term does not depend on S or a, so we can ignore it when maximizing the likelihood. Additionally, the ½ in front of the sum can be ignored, as it is a constant factor that does not affect the maximizer. Finally, we can more concisely state this as a minimization problem instead of a maximization problem by multiplying by −1. This means that maximizing the likelihood is equivalent to minimizing the function:

$$f ( S , a ) = {\sum }_{k = 1}^{K} {( {S}_{k}^{detrend} - {a}_{k} S )}^{T} {\sum }^{- 1} ( {S}_{k}^{detrend} - {a}_{k} S ) . \qquad ( 3.1 )$$

Now, denote by L the matrix found by taking the Cholesky Decomposition of Σ⁻¹, so Σ⁻¹=LL^{T}. Then,

$$\begin{matrix}f ( S , a ) = {\sum }_{k = 1}^{K} {( {S}_{k}^{detrend} - {a}_{k} S )}^{T} {\sum }^{- 1} ( {S}_{k}^{detrend} - {a}_{k} S ) \\ = {\sum }_{k = 1}^{K} {( {S}_{k}^{detrend} - {a}_{k} S )}^{T} {LL}^{T} ( {S}_{k}^{detrend} - {a}_{k} S ) \\ = {\sum }_{k = 1}^{K} {( {L}^{T} ( {S}_{k}^{detrend} - {a}_{k} S ) )}^{T} ( {L}^{T} ( {S}_{k}^{detrend} - {a}_{k} S ) ) \\ = {\sum }_{k = 1}^{K} {‖ {L}^{T} ( {S}_{k}^{detrend} - {a}_{k} S ) ‖}_{2}^{2} \\ = {‖ {S}^{detrend} L - {aS}^{T} L ‖}_{F}^{2}\end{matrix}$$

where ∥⋅∥_{F} denotes the Frobenius norm and S^{detrend} is the K×T matrix formed by stacking the vectors Sₖ^{detrend} row-wise. This is not a straightforward optimization problem to solve, but noting that aS^{T}L is a rank-1 matrix, the Schmidt, Misrky, Eckart-Young Theorem states that the rank-1 matrix that minimizes the Frobenius norm of the difference between it and S^{detrend}L is the matrix σ₁u₁v₁^{T}, where σ₁ is the largest singular value of S^{detrend} L and u₁ and v₁ are the corresponding left and right singular vectors. Therefore, we have the following equation relating the optimal estimates S and a to the singular value decomposition of S^{detrend} L:

$$\hat{a} {\hat{S}}^{T} L = {σ}_{1} {u}_{1} {v}_{1}^{T} \\ \hat{a} {\hat{S}}^{T} = {σ}_{1} {u}_{1} {v}_{1}^{T} {L}^{- 1}$$

which implies that the optimal estimates are:

$$\hat{S} = \frac{{( {L}^{- 1} )}^{T} {v}_{1}}{{‖ {( {L}^{- 1} )}^{T} {v}_{1} ‖}_{2}} \\ \hat{a} = {‖ {( {L}^{- 1} )}^{T} {v}_{1} ‖}_{2} {σ}_{1} {u}_{1} .$$

Furthermore, this estimate is unique up to a sign change on all but a set of measure zero. It is often convenient to choose the sign so that â is mostly nonnegative.

It is often the case that it is desirable to allow the pulse shape to vary over time. In fact, in the case of trying to estimate glucose from PPG signals, we expect that there may be some variability in the shape of the pulse over time and we want to be able to detect that change. If the pulse shape does not change too quickly, then the pulse shape is approximately constant over a window. We can therefore perform the same analysis as above, but over a sliding window that has a length of K pulses.

One additional consideration, however, is the possibility of weighting different pulses in a window. If the pulse shape changes slowly, then the pulse shape in the middle of the window is likely to be a better estimate of the pulse shape than the pulse shape at the edges of the window.

Suppose that we have a window of K pulses and that we wish to apply K weights

$${\{ {w}_{k} \}}_{k = 1}^{K}$$

to the pulses. We can start with a modified version of equation (3.1):

$$f ( S , a ) = {\sum }_{k = 1}^{K} {{w}_{k} ( {S}_{k}^{detrend} - {a}_{k} S )}^{T} {\sum }^{- 1} ( {S}_{k}^{detrend} - {a}_{k} S ) .$$

Notice that applying a weight to pulse k has the direct interpretation of scaling the covariance matrix of pulse k by 1/wₖ because

$${w}_{k} {\sum }^{- 1} = {( \frac{1}{{w}_{k}} \sum )}^{- 1} .$$

Therefore, a lower weight imparts greater uncertainty in the pulse shape and amplitude. Following the same process as in the proof for Theorem 3.1 but instead starting from the weighted equation yields:

$$\begin{matrix}f ( S , a ) = {\sum }_{k = 1}^{K} {w}_{k} {‖ {L}^{T} ( {S}_{k}^{detrend} - {a}_{k} S ) ‖}_{2}^{2} \\ = {\sum }_{k = 1}^{K} {‖ \sqrt{{w}_{k}} {L}^{T} ( {S}_{k}^{detrend} - {a}_{k} S ) ‖}_{2}^{2} \\ = {‖ {WS}^{detrend} L - {WaS}^{T} L ‖}_{F}^{2}\end{matrix}$$

Where W is the K×K matrix:

$$W = [ \begin{matrix}\sqrt{{w}_{1}} & 0 & … & 0 \\ 0 & \sqrt{{w}_{2}} & … & 0 \\ ⋮ & ⋮ & ⋱ & ⋮ \\ 0 & 0 & … & \sqrt{{w}_{K}}\end{matrix} ] .$$

Thus, weighting the pulses in the loss function can be accomplished by simply scaling the rows of the matrix S^{detrend} by the square root of the weight before taking the SVD. Then, the estimates for S and a are:

$$\hat{S} = \frac{{( {L}^{- 1} )}^{T} {v}_{1}}{{‖ {( {L}^{- 1} )}^{T} {v}_{1} ‖}_{2}} \\ \hat{a} = {‖ {( {L}^{- 1} )}^{T} {v}_{1} ‖}_{2} {σ}_{1} {W}^{- 1} {u}_{1}$$

where the SVD is taken of the matrix WS^{detrend}L.

Because this method is on a sliding window of length K, note that for each pulse we are actually given K different estimates for pulse shape and amplitude, one for each window that contains that pulse. The pulse shapes can be interpreted as the estimate for the pulse shape under the assumption that the pulse shape is constant over a particular window. The amplitudes can be interpreted as the estimate for the amplitude under the assumption that a particular pulse shape is the correct pulse shape. Regardless, because there are K estimates for pulse shape and amplitude, it is necessary to choose some method for agglomerating these estimates. The simplest and computationally cheapest way to do this is to simply take the pulse shape and amplitude that is found when the window is centered on the pulse.

This is the method employed in the example presented herein, and can therefore be used in various implementations based thereon. However, after having received the benefit of this disclosure, other methods will be apparent to those of ordinary skill in the art.

For example, another simple and relatively cheap method is to take the (weighted) average of the pulse shapes and amplitudes over all windows that contain a particular pulse. An example of a full algorithm under this method is provided below as “Algorithm 2,” otherwise referred to herein as “Phase-Lock Averaging over a Sliding Window.”

Stated generally, Algorithm 2 comprises the following steps, which may, but need not always, be performed in the precise sequence listed below.

First, the signal, such as a heartbeat or other physiological signal, may be detrended. The detrended signal may, again, be split into vectors, such as split into N K×T period-length vectors

$${\{ {S}_{n}^{detrend} \}}_{n = 1}^{N} .$$

Each matrix may then be scaled. For example, the k-th row of each matrix may be scaled by √wₖ to get

$${\{ {WS}_{n}^{detrend} \}}_{n = 1}^{N} .$$

For n=1 to N, Algorithm 1 may be performed on WSₙ^{detrend}, which may provide estimates for Ŝ and â. For n=1 to N, âₖ may be multiplied by 1/√wₖ to recover estimates for the amplitudes.

Each pulse may now have K estimates for pulse shape and amplitude. These estimates may then be agglomerated using a desired method to obtain one estimate for each pulse.

The above algorithm is a fairly naive way to estimate the pulse shape and amplitude when the pulse shape is allowed to vary over time. Because of this, one might wonder if there is any case in which it gives an optimal estimate. There is, in fact, at least one example of a case in which Algorithm 2 gives the maximum likelihood estimate.

Algorithm 2 is best thought of as an analog to a moving average, but instead of finding a trend in a signal, it is finding a ‘trending’ pulse shape. In the same way that taking a moving average gives an unbiased estimate of the trend in a signal only in very specific circumstances, Algorithm 2 gives an unbiased estimate of the pulse shape and amplitude only in very specific circumstances. Despite this, taking the moving average to estimate a trend is a valuable tool in many situations, and the same can be said for Algorithm 2.

Theorem 3.2: Suppose that the pulse shape and amplitude of a signal can be written as

$${S}_{n}^{detrend} = {a}_{n} {S}_{n} + {ε}_{n} \\ {a}_{n} {S}_{n} = {a}_{n - 1} {S}_{n - 1} + {δ}_{n}$$

where εₙ˜N (0, σ²_{ε}I) are i.i.d., δₙ˜N (0, σ²_{δ}I) are i.i.d. and all εₙ and δₙ are independent.

The Phase-Lock Averaging Estimator Over A Sliding Window (Algorithm 2) is the maximum likelihood estimator over a window of length K+1 when the weights are chosen to be

$${W}_{k} = \frac{1}{1 + \vert k \vert \frac{{σ}_{δ}^{2}}{{σ}_{ε}^{2}}}$$

and the agglomeration method is to take the estimate when the window is centered on the pulse.

Suppose that we want to estimate the pulse shape and amplitude at time n. To find the maximum likelihood estimator, first note that we can rewrite the

$${S}_{i}^{detrend}$$

vectors as:

$$⋮ \\ {S}_{n - 2}^{detrend} = {a}_{n} {S}_{n} + {ε}_{n - 2} - {δ}_{n} - {δ}_{n - 1} \\ {S}_{n - 1}^{detrend} = {a}_{n} {S}_{n} + {ε}_{n - 1} - {δ}_{n} \\ {S}_{n - 2}^{detrend} = {a}_{n} {S}_{n} + {ε}_{n} \\ {S}_{n + 1}^{detrend} = {a}_{n} {S}_{n} + {ε}_{n - 1} + {δ}_{n + 1} \\ {S}_{n + 2}^{detrend} = {a}_{n} {S}_{n} + {ε}_{n + 2} - {δ}_{n + 2} + {δ}_{n + 1} \\ ⋮$$

And, in general,

$${S}_{n + k}^{detrend} = {a}_{n} {S}_{n} + {γ}_{n + k} ,$$

where we define

$${γ}_{n + k} = \{ \begin{matrix}{ε}_{n + k} + {\sum }_{i = 1}^{k} {δ}_{n + i} & if k > 0 \\ {ε}_{n} & if k = 0 \\ {ε}_{n + k} - {\sum }_{i = k}^{- 1} & if k < 0\end{matrix} .$$

Notice that each γₙ₊ₖ is the sum of independent Gaussian random vectors and is therefore itself Gaussian. Furthermore, we can calculate the mean and covariance of γₙ₊ₖ. Its mean is the sum of the means of its constituent random vectors, which is zero. Its covariance is the sum of the covariances of its constituent random vectors, which is

$${σ}_{ε}^{2} I + \vert k \vert {σ}_{δ}^{2} I = ( {σ}_{ε}^{2} + \vert k \vert {σ}_{δ}^{2} ) I .$$

Now, we can write the likelihood below:

$$L ( {s}_{n} , {a}_{n} ) = {{\prod }_{k = - K / 2}}^{K / 2} \frac{1}{\sqrt{{( 2 π )}^{T} {( {σ}_{c}^{2} + \vert k \vert {σ}_{δ}^{2} )}^{T}}} exp ( - \frac{{( {S}_{n + k}^{detrend} - {a}_{n , k} {S}_{n} )}^{T} ( {S}_{n + k}^{detrend} - {a}_{n , k} {S}_{n} )}{2 ( {σ}_{ε}^{2} + \vert k \vert {σ}_{δ}^{2}} ) .$$

Following the same steps as in Theorem 3.1, we can maximize the likelihood by minimizing the function:

$$\begin{matrix}f ( {S}_{n} , {a}_{n} ) = {\sum }_{k = - K / 2}^{K / 2} \frac{1}{{σ}_{ε}^{2} + \vert k \vert {σ}_{δ}^{2}} {( {S}_{n + k}^{detrend} - {a}_{n , k} {S}_{n} )}^{T} ( {S}_{n + k}^{detrend} - {a}_{n , k} {S}_{n} ) \\ = {\sum }_{k = - K / 2}^{K / 2} {‖ \frac{1}{\sqrt{{σ}_{ε}^{2} + \vert k \vert {σ}_{δ}^{2}}} ( {S}_{n + k}^{detrend} - {a}_{n , k} {S}_{n} ) ‖}_{2}^{2} \\ = {\sum }_{k = - K / 2}^{K / 2} {‖ \frac{1}{\sqrt{1 + \vert k \vert \frac{{σ}_{δ}^{2}}{{σ}_{ε}^{2}}}} ( {S}_{n + k}^{detrend} - {a}_{n , k} {S}_{n} ) {σ}_{ε}^{- 1} ‖}_{2}^{2} \\ = {‖ {WS}_{n}^{detrend} {σ}_{ε}^{- 1} - {Wa}_{n} {S}_{n}^{T} {σ}_{ε}^{- 1} ‖}_{F}^{2}\end{matrix} ,$$

$$where$$

$$W = [ \begin{matrix}\frac{1}{\sqrt{1 + \frac{K}{2} \frac{{σ}_{δ}^{2}}{{σ}_{ε}^{2}}}} & 0 & … & 0 \\ 0 & \frac{1}{\sqrt{1 + ( \frac{K}{2} - 1 ) \frac{{σ}_{δ}^{2}}{{σ}_{ε}^{2}}}} & … & 0 \\ ⋮ & ⋮ & ⋱ & ⋮ \\ 0 & 0 & … & \frac{1}{\sqrt{1 + \frac{K}{2} \frac{{σ}_{δ}^{2}}{{σ}_{ε}^{2}}}}\end{matrix} , ]$$

and Sₙ^{detrend} is the (K+1)×T matrix formed by stacking the vectors

$${\{ {S}_{n + k}^{detrend} \}}_{k = - K / 2}^{K / 2}$$

row-wise.

Applying the Schmidt, Mirsky, Eckart-Young Theorem as in Theorem 3.1, we find that the optimal estimates for Sₙ and aₙ are:

$${\hat{S}}_{n} = {v}_{1}$$

$${\hat{a}}_{n} = {σ}_{1} {W}^{- 1} {u}_{1}$$

where the u₁, σ₁, and v₁ come from the SVD of WSₙ^{detrend}. Finally, we estimate the amplitude at time n to be âₙ, K/2+1 (the agglomeration method being to simply choose the amplitude at the center of the window). This estimate is exactly what Algorithm 2 gives.

While the theory for the Phase-Lock Averaging Estimator is elegant, there are several practical considerations that need to be made to apply it to real data. These considerations are discussed below.

A person's heart rate changes depending on the time of day and level of physical activity. This means that the pulse shape is not a periodic signal. Failing to take heart rate variability into account will likely lead to poor estimates of the pulse shape and amplitude.

To address this issue, a technique and/or algorithm referred to herein as “Dynamic Resampling” may be used, as described in William Richard Terry, “Modeling blood-glucose levels via near-infrared spectroscopy,” BYU Electronic Theses and Dissertations, 2024, which paper is hereby incorporated herein by reference in its entirety.

The basic idea behind this technique is as follows:
(i) Use a peak-finding algorithm to find the peaks of the signal.
(ii) Use the found peaks to separate the signal into individual pulses.
(iii) Resample each pulse to a common length.

The peak-finding method may therefore be of significance to the success of Dynamic Resampling, and by extension, the Phase-Lock Averaging Estimator. It is not a straightforward problem, however, and there are many methods that have been devised to find peaks in PPG signals.

In this disclosure, the peak-finding algorithm will not be discussed, and we will instead assume that the peaks have been found and that the signal has been separated into individual pulses.

In this disclosure, we also do not attempt to quantify the effect of errors in the peak-finding algorithm, but experience has shown that a poor peak-finding algorithm can be detrimental to the estimate of the pulse shapes. One way to mitigate this is to take a multi-modal approach. ECG signals have very strong peaks, so it is easy to segment the signal into individual pulses. By syncing the PPG signal with the ECG signal, we can use the ECG signal to segment the PPG signal into individual pulses.

There are likewise many methods for resampling a signal to a common length. Many of these methods, however, can be written simply as a linear transformation of the signal.

For example, the method that our implementation of Dynamic Resampling uses is to interpolate the signal at a particular set of points. The device takes samples at a rate of 100 Hz, and a natural choice for the desired heart rate is 60 beats per minute, or 1 Hz, as this is near the average heart rate of a human at rest. This means that however long a pulse is found to be in the original signal, the goal is to resample it so that it is 100 samples long. Additionally, our exploration of the data showed that the length of the power stroke of the pulse is approximately constant regardless of the heart rate, and that it is around 35 samples long.

This led us to the following, exemplary resampling procedure:
(i) Keep the first 35 samples of the pulse as they are.
(ii) Interpolate the remaining samples at equal time intervals to get 65 samples.

While not obvious at first, this is a linear transformation applied to the vector of samples that make up the pulse. If the pulse has length L>35, but we desire it to have length T=100, then the Dynamic Resampling matrix is the T×L matrix M:

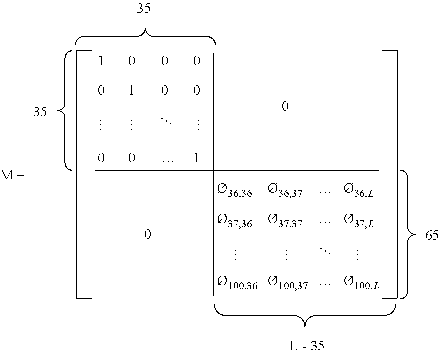

where the ϕ_{i,j} are the coefficients from the interpolation function. In the case of linear interpolation, which is used by our implementation of Dynamic Resampling, each row of M has at most two non-zero entries, which sum to 1.

Most resampling methods can, in fact, be written as a linear transformation (not just Dynamic Resampling). This makes them very amenable to the Phase-Lock Averaging Estimator. Suppose that M is a resampling matrix. Next, suppose that a single pulse can be written in vector form as:

$${S}_{n}^{pulse} = {a}_{n} S + {ε}_{n}$$

where Sₙ^{pulse} has length L and εₙ˜N (0, Σ^{pulse}). We desire to resample the pulse to length T. We can use the resampling matrix to write the resampled pulse as:

$$\begin{matrix}{S}_{n}^{resampled} = {MS}_{n}^{pulse} \\ = M ( aS + ε ) \\ = aMS + M ε\end{matrix} .$$

Notice that this has the same general form expected for the Phase-Lock Averaging Estimator. The only difference is that the pulse shape is now the resampled pulse shape MS and the noise is the resampled noise Mε. Furthermore, the resampled noise is still Gaussian because each entry is the sum of Gaussian random variables with a Gaussian joint distribution. We can calculate the mean and covariance of the resampled noise:

$$\begin{matrix}{μ}^{resampled} = 𝔼 [ M ε ] \\ = M 𝔼 [ ε ] \\ = 0\end{matrix}$$

$$\begin{matrix}{Σ}^{resampled} = 𝔼 [ ( M ε - {μ}^{resampled} ) {( M ε - {μ}^{resampled} )}^{T} ] \\ = 𝔼 [ M {εε}^{T} {M}^{T} ] \\ = M 𝔼 [ {εε}^{T} ] {M}^{T} \\ = M {Σ}^{pulse} {M}^{T}\end{matrix} .$$

This computation gives an argument for a case in which one might choose a covariance matrix for the noise that is not simply a multiple of the identity matrix.

If we assume that the noise is white noise, then the covariance matrix that follows is Σ^{pulse}=σ²I for some σ² (all entries of εₙ are independent and identically distributed). The covariance matrix of the resampled noise is then Σ^{resampled}=σ²MM^{T}.

One notable potential issue is that if L<T, then the covariance matrix Σ^{resampled}=MΣ^{pulse}M^{T} is singular. However, this is not an issue because technically what we are interested in is the matrix E_{M}[MΣ^{pulse}M^{T}] as each individual pulse is resampled differently. This matrix is likely not singular, so the Phase-Lock Averaging Estimator can still be applied using the matrix E_{M}[MΣ^{pulse}M^{T}], which can be estimated over many pulses by averaging the matrices MΣ^{pulse}M^{T}.

While collecting real-world data, there is a lot that can go wrong. For example, a sudden change in lighting can corrupt the signal because the ambient light has changed. Or, the device may have been moved slightly, causing artifacts. One way to deal with this is to mask out the corrupted data.

In some embodiments and implementations, this can be accomplished by setting the rows of the S^{detrend} matrix that contain corrupted pulses to zero (or simply removing them from the matrix). As explained above in connection with the discussion regarding Phase-Lock Averaging over a Sliding Window, scaling the rows of the S^{detrend} matrix is equivalent to weighting the pulses in the loss function by the square of the scaling factor. Therefore, setting the rows to zero is equivalent to giving the corrupted pulses a weight of zero in the loss function.

In the examples presented herein, three quality metrics were used to determine if a pulse is corrupted.

First, the Signal Quality Index (SQI) is a measure of how well the pulse aligns with its neighbors. It is calculated by taking the Pearson correlation coefficient between the pulse and its neighbors.

Second, the Pulse Quality Index (PQI) is a rough measure of how much of the power of the signal is contained in the pulse. Instead of being calculated for a single pulse like the SQI, it is computed over a short time window of the signal, containing several pulses. The PQI is defined by taking the ratio of the power contained in the three most powerful frequencies to the power of the remaining frequencies.

Third, the Maximum Absolute Deviation (MAD) is a simple measure of how much the signal deviates from the mean. The mean of a window of the signal is calculated, and then the maximum absolute deviation from this mean is taken over a pulse. This value is assigned to the pulse.

One can then set a threshold range for each of these metrics, and, if a pulse falls outside of the threshold range for any of the metrics, it can be masked out.

In addition to pulse corruption, described above, there is at least one other source of missing data that may find benefit to address. As explained above, some devices periodically take an ambient light measurement. During

this time, no pulse is visible in the signal, so several pulses are dropped. Using the heart rate, we can estimate the number of pulses that are missing, but there is no data to fill in the missing pulses.

Many downstream algorithms are unable to function properly if there are missing data points. To use these algorithms, it may be necessary or at least desirable to fill in missing data. Ways that this can be done include linear interpolation, nearest neighbor imputation, mean imputation, or simply removing the data. However, these methods fall short because they either do not take the pulse shape into account, or do not take the time series nature of the data into account. When pulse shape is an important feature of the data (as it is in our research), it may be useful to use a method that takes the pulse shape into account.

Phase-Lock Averaging can be used as a data imputation method. The idea is to use the pulse shape and amplitude of the pulses that are present to estimate the pulse shape and amplitude of the pulses that are missing. By simply centering a window on the missing pulse and masking the corrupted data, one can estimate the pulse shape of the missing pulse. Once the pulse shape is estimated, all that is needed is to estimate the amplitude.

Recall that Phase-Lock Averaging gives an estimate for the amplitude of all of the pulses in the window under the assumption that the estimated pulse shape is correct. Therefore, we can assign an amplitude to the imputed pulse by taking the (weighted) average of the amplitudes of the other pulses in the window. This method provides a way to impute missing data that takes the pulse shape into account while smoothly filling in the missing data.

In some implementations, such a method may be summarized as follows:
(i) Mask out the missing pulse(s).
(ii) Center a window on the missing pulse.
(iii) Use the Phase-Lock Averaging Estimator to estimate the pulse shape of the missing pulse.
(iv) Using the estimates for the amplitude of the other pulses in the window, estimate the amplitude of the missing pulse.
(v) Fill in the missing pulse with the estimated pulse shape and amplitude.

This can be performed seamlessly in Algorithm 2 by simply adding a step to impute missing data. In some implementations of the algorithm, this may be done.

One downside that Phase-Lock Averaging has is that it is computationally expensive. Surprisingly, though, under the assumption that the pulse shape is constant over the entire length of the signal, the Phase-Lock Averaging Estimator can actually be faster than Fourier methods. If N is the number of samples taken by the device, then Fourier methods can be done in O(N log N) time by taking the Fast Fourier Transform (FFT) of the signal. Phase-Lock Averaging with Constant Pulse Shape, however, can be done in O(NT) time, where T is the length of the pulse, which is usually taken to be constant at 100 samples, and is much smaller than N. This is because the limiting factor is the SVD, which for an m×n matrix has a time complexity of O(min(n²m, nm²)). The matrix that we are taking the SVD of is a K×T matrix, where K≤N/T. Thus, the time complexity is O(KT²)=O(NT).

Despite this, in practical applications, Phase-Lock Averaging will usually always be slower than any Fourier Method, even if a method is found to speed up the calculation of the many overlapping SVDs in Algorithm 2. This is simply due to the fact that in order to perform Phase-Lock Averaging, it is typical to find peaks in the signal, which is often done by first applying a filter. Then, we need to resample the pulses to a common length, which is an operation that could be as slow as O(N²) because it can be thought of as a matrix multiplication with a matrix of the size N×N (it is usually able to be done much quicker, however). Only at that point are we able to apply either Algorithm 1 or Algorithm 2.

Still, if the pulse shape is constant over the entire signal, all of the pulses are known ahead of time, and the signal is long enough, then Algorithm 1 can be faster than Fourier methods.

In most practical applications, Algorithm 1 is not applicable because the pulse shape is not constant over the entire signal. This means that Algorithm 2 must be used, which has time complexity O(NKT), where K in this case is the number of pulses contained in the window. This is because, like Algorithm 1, the limiting factor is the SVD, which has time complexity O(KT²). However, here the SVD is taken of a K×T matrix and must be performed approximately N/T times because the window is slid over the signal. This makes the time complexity O(NKT). While K and T are taken to be constant, making the algorithm asymptotically O(N), the constant factor is very large. In our research, K=301 and T=100, so KT=30100. The signal would have to be very long indeed for the constant factor to be small enough for Phase-Lock Averaging to be faster than Fourier methods.

One might wonder, however, if the constant factor might be reduced by using a more efficient method to calculate the SVD. After all, we do not need the full SVD, only the first singular value and its corresponding singular vectors.

One method to do this that we found to be effective is the Randomized SVD. The basic idea is surprisingly simple. Suppose that we have an m×n matrix A that we wish to compute the SVD of, but we only need the first k singular values/vectors. Instead of computing the full SVD of A, we chose an integer [symbol] >k and a random m× [symbol] orthogonal matrix Q with [symbol] <<m and compute B=Q^{T}A. This gives a matrix B that is [symbol] ×n. Then, we can compute the SVD of B and use the singular vectors of B to construct the singular vectors of A. We compute B=ŨΣVT and set U=QŨ. Then, A≈UΣV^{T} is a rank- [symbol] approximation of the SVD of A. Using the rank- [symbol] approximation, we can compute the first k singular values and vectors of A to surprising accuracy.

There are many choices that can be made to optimize the process. However, in certain preferred embodiments and implementations, the algorithm follows the same basic plan:
(i) Choose an orthogonal matrix Q such that A≈QQ^{T}A. This is a projection onto a rank- [symbol] subspace of A. Using Q, compute the matrix B=Q^{T}A.
(ii) Use a standard algorithm to compute the SVD of B. Then, using this decomposition B=ŨΣV^{T}, compute A≈QŨΣV^{T}.

The computational bottleneck of the algorithm is typically step (i). In some cases, the choice may be as follows:
(i) We draw a matrix Ω from the distribution of matrices in which all entries are i.i.d. standard normal random variables.
(ii) We compute the matrix Y=AΩ.
(iii) We find the QR decomposition of Y=QR.
(iv) We use this Q to compute B=Q^{T}A.

This is not the most computationally efficient method that can be used to compute the Randomized SVD, but it has the significant advantage that it is easy to implement and that its accuracy can be bounded. For dense matrices, a structured matrix like the FFT may be used for Q because the multiplication Q^{T}A can be computed cheaply. However, using such a matrix would require a more complicated implementation and does not come with the same guarantees of accuracy that the Gaussian matrix does.

The following bound for the accuracy of the above method may be given:

$$‖ A - {QQ}^{T} A ‖ \leq 10 √ \frac{2}{π} {max}_{i = 1 … ℓ} ‖ ( A - {QQ}^{T} A ) {ω}_{i} ‖$$

with probability at least 1- [symbol] .

In some embodiments and implementations, [symbol] may be chosen to be about 5 bigger than k. Because of this (and being slightly more conservative), since our desired k is k=1, in the example presented herein, [symbol] =7 was chosen, which gives very high probability that the error is small, while providing a significant speedup.

The computational complexity of this algorithm can be broken down as follows:

$$Drawing Ω \sim O ( n ℓ ) \qquad ( i )$$

$$Multiplying Y = A Ω \sim O ( mn ℓ ) \qquad ( ii )$$

$$Finding the QR decomposition of Y \sim O ( mn ℓ ) \qquad ( iii )$$

$$Multiplying B = {Q}^{⊤} A \sim O ( mn ℓ ) \qquad ( iv )$$

$$Computing the SVD of B = \tilde{U} Σ {V}^{⊤} \sim O ( n {ℓ}^{2} ) \qquad ( v )$$

$$Multiplying U = Q \tilde{U} \sim O ( mn ℓ ) \qquad ( vi )$$

Thus, this method is bounded by O(mn [symbol] ), which is much faster than the O(mn min(m, n)) time complexity of the full SVD.

Using the Randomized SVD algorithm to calculate the SVD in Phase-Lock Averaging translates to a time complexity of O(N [symbol] ) for Algorithm 1 and O(NK [symbol] ) for Algorithm 2. To compare these two methods, Algorithm 2 was run on a 2021 MacBook Pro 14-inch with an Apple M1 Pro chip and 32 GB of RAM. We used a signal of length N=801200 and K=201. Additionally, there were 30 wavelength channels in the signal, so the algorithm was run 30 times. Without the Randomized SVD, the algorithm took 14 minutes and 15 seconds to finish. With the Randomized SVD, the algorithm took 25 seconds to finish. Treating the deterministic method as the ground truth, the MSE of the Randomized SVD method was 14×10⁻⁸, small enough to be negligible.

Phase-Lock Averaging gives the Maximum Likelihood Estimator of the pulse shape and amplitude under the assumption that the pulse shape is constant over the window. However, the way that the problem is set up also allows for the incorporation of prior information about the pulse shape. Doing this changes the problem from finding the MLE to finding the MAP (Maximum A Posteriori) Estimator. Recall Bayes' Rule:

$$P ( S , a \vert {S}_{1}^{detrend} , … , {S}_{K}^{detrend} ) - \frac{P ( {S}_{1}^{detrend} , … , {S}_{K}^{detrend} \vert S , a ) PP ( S , a )}{P ( {S}_{1}^{detrend} , … , {S}_{K}^{detrend} )} .$$

Because P(S₁^{detrend}, . . . , Sₖ^{detrend}) is constant with respect to S and a, we can maximize the numerator to find the MAP Estimator. Notice also that P(S₁^{detrend}, . . . , Sₖ^{detrend}|S, a) is the same likelihood as from Theorem 3.1. Therefore, taking the log of the numerator, we have:

$$log P ( {S}_{1}^{detrend} , … , {S}_{K}^{detremd} \vert S , a ) P ( S , a ) = ℓ ( S , a \vert {S}_{1}^{detrend} , … , {S}_{K}^{detrend} ) + log P ( S , a ) .$$

This suggests that we can minimize the function

$$f ( S , a ) = {‖ {S}^{detrend} L - {aS}^{T} L ‖}_{F}^{2} - 2 log P ( S , a )$$

to find the MAP Estimator. In other words, adding prior information is as simple as adding a regularization term to the loss function.

Unfortunately, because the optimization problem is solved by computing the SVD of a matrix, it is not straightforward to add a regularization term to the loss function. However, if the prior distributions for pulse shape and amplitude are restricted to specific distributions, it is still possible to find the MAP Estimator.

The fact that incorporating a prior is equivalent to adding a regularization term immediately suggests a way to add a prior for pulse shape: If we can find a way to make the regularization term take the same form as the data, adding the regularization term would be equivalent to adding an extra row to the matrix that we take the SVD of. We can do this by adding an extra parameter α and assuming that −2 log P(S, a)=(S^{prior}−αS)^{T}Σ⁻¹

(S^{prior}−αS). Then, we get the following loss function:

$${f ( S , a , α ) = {S}^{prior} - αS )}^{T} {\sum }^{- 1} ( {S}^{prior} - αS ) + {\sum }_{k = 1}^{K} {( {S}_{k}^{detrend} - {α}_{k} S )}^{T} {\sum }^{- 1} ( {S}_{k}^{detrend} - {α}_{k} S ) .$$

From here, it is clear that we can minimize the same way as was done in Theorem 3.1. Namely, we construct a matrix

$${S}^{stack} = [ \begin{matrix}\frac{{S}^{{prior}^{T}}}{{S}_{1}^{{detrend}^{T}}} \\ ⋮ \\ {S}_{K}^{{detrend}^{T}}\end{matrix} ]$$

And the function to minimize becomes:

$$f ( S , \text{?} ) = {‖ {S}^{stack} L - \text{?} SL ‖}_{F}^{2}$$

$$\text{?} \text{indicates text missing or illegible when filed}$$

where ã^{T}=[α|α₁ . . . α_{K}].

This function can be minimized by taking the SVD of SstackL, the same way as was done in Theorem 3.1. This gives the estimates:

$$\hat{S} = \frac{{( {L}^{- 1} )}^{T} {v}_{1}}{{‖ {( {L}^{- 1} )}^{T} {v}_{1} ‖}_{2}}$$

$$\text{?} = {‖ {( {L}^{- 1} )}^{T} {v}_{1} ‖}_{2} {σ}_{1} {u}_{1}$$

$$\text{?} \text{indicates text missing or illegible when filed}$$

where the SVD is taken of S^{stack}L. The optimal estimate for a can then be extracted from

$$\text{?} since \text{?} = [ \hat{a} \vert \hat{{a}_{1}} … {\hat{a}}_{K} ] .$$

$$\text{?} \text{indicates text missing or illegible when filed}$$

For clarity, the distributional assumptions for the prior for S and a were not stated. We address that now. To ensure that −2 log P(S, a)=(S^{prior}−αS)^{T}Σ⁻¹(S^{prior}−αS), we first assume that S and a are independent. Then, we can write the posterior probability

$$P ( S , a \vert {S}_{1}^{detrend} , … , {S}_{K}^{detrend} ) ∝ P ( {S}_{1}^{detrend} , … , {S}_{K}^{detrend} \vert S , a ) P ( S ) P ( a ) .$$

Next, we want to eliminate P(a) from the expression. Because we want to make minimal restrictive assumptions about a, we will choose a positive real number M and assume that each aₖ is independent with aₖ˜Unif[−M, M]. In order for this prior to not interfere with a, we will assume that M is large enough that it covers all realistic values of aₖ. The result is that P(a) can then be ignored, since it is a constant. We get

$$P ( S , a \vert {S}_{1}^{detrend} , … , {S}_{K}^{detrend} ) ∝ P ( {S}_{1}^{detrend} , … , {S}_{K}^{detrend} \vert S , a ) P ( S ) ,$$

For all realistic values of a. Finally, we will assume that

$$P ( S ) ∝ exp ( - \frac{1}{2} {( {S}^{prior} - αS )}^{T} {\sum }^{- 1} ( {S}^{prior} - αS ) )$$

where, for each S, α is fixed to maximize exp (−½(S^{prior}−αS)^{T}Σ⁻¹(S^{prior}−αS). This form of the PDF is shown so that it is clear why this distribution is a convenient distribution to choose for the prior for S. However, to show that this is, in fact, a distribution for S, we show that α can have only one value for each S.

We will fix S and maximize exp (−½ (S^{prior}−αS)^{T}Σ⁻¹(S^{prior}−αS) by minimizing the negative logarithm. Taking the derivative with respect to α and setting it equal to 0, we have

$$0 = {( {S}^{prior} - α S )}^{T} {\sum }^{- 1} S$$

$$0 = {S}^{{prior}^{T}} {\sum }^{- 1} S - α {S}^{T} {\sum }^{- 1} S$$

$$α {S}^{T} {\sum }^{- 1} S = {S}^{{prior}^{T}} {\sum }^{- 1} S$$

$$α = \frac{{S}^{{prior}^{T}} {\sum }^{- 1} S}{{S}^{T} {\sum }^{- 1} S} ,$$

which we can substitute into the PDF to remove α. If we make the additional assumptions that Σ=κ⁻¹I and ∥S^{prior}=1∥, then α=S^{priorT}S, and

$$\begin{matrix}P ( S ) ∝ exp ( - \frac{1}{2} ( {S}^{{prior}^{T}} {S}^{prior} - 2 α {S}^{{prior}^{T}} S + {S}^{T} S ) ) \\ = exp ( κα {S}^{{prior}^{T}} S - κ ) \\ ∝ exp ( {κ ( {S}^{{prior}^{T}} S )}^{2} ) .\end{matrix}$$

This distribution is remarkably similar to the von Mises-Fisher Distribution, which is one of the most fundamental distributions in directional statistics:

$$P ( S ) ∝ exp ( κ {S}^{{prior}^{T}} S ) . \qquad ( von Mises - Fisher Distribution )$$

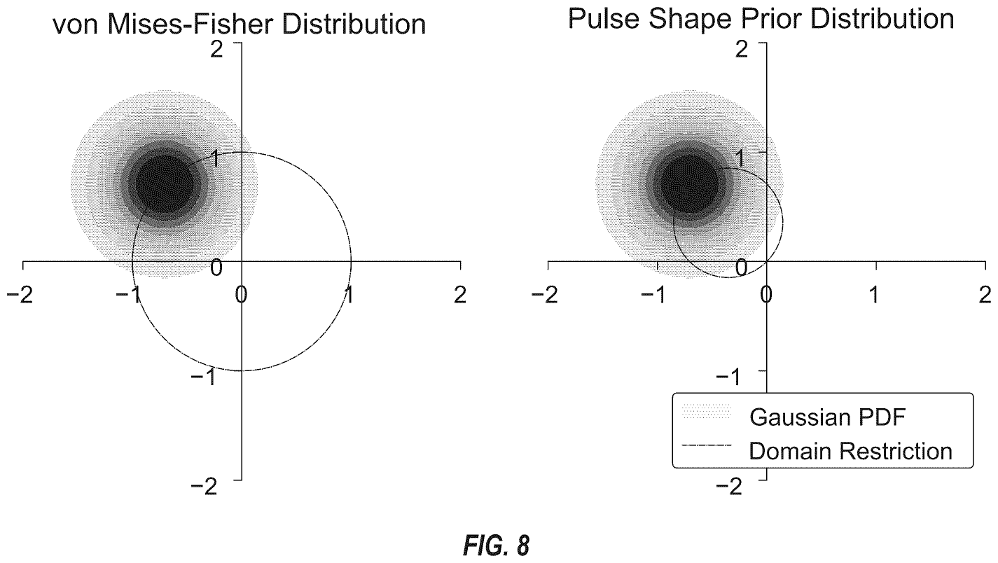

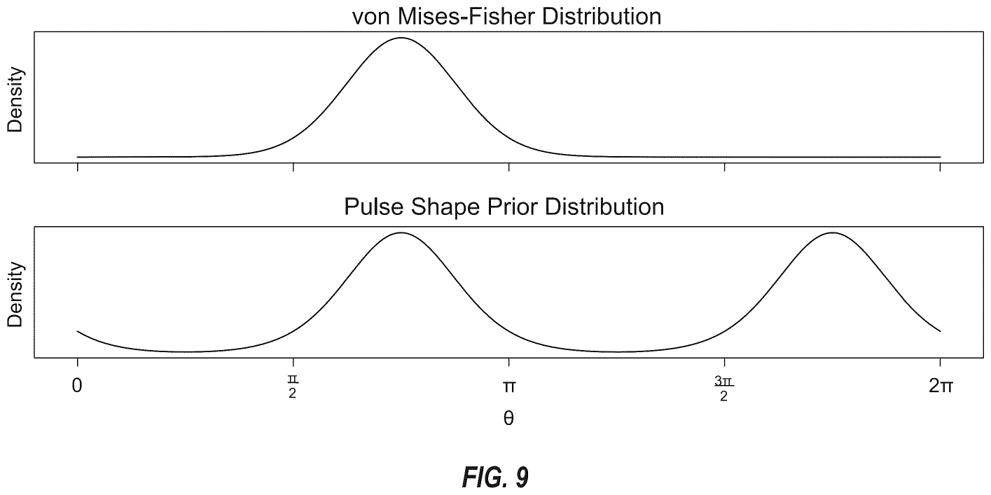

The difference is that the von Mises-Fisher distribution is defined by restricting the domain of a Gaussian distribution in Rn to the unit sphere, while the pulse shape prior distribution is a Gaussian restricted to the curve that maximizes the functional part of the Gaussian PDF (See FIG. 8). A key property of the pulse shape prior distribution is that P(S)=P(−S) for all S in its support (See FIG. 9). This is not true for the von Mises-Fisher distribution. The property is required because PLA gives estimates that are only unique up to sign change.

The ability to incorporate a prior for the pulse shape leads immediately to a much faster way to perform PLA in real time on a signal with a changing pulse. The idea is to track a pulse shape and perform a Bayesian update to the pulse shape as new data becomes available. Doing this with the weighting idea, as discussed above, allows us to include a confidence factor in the Bayesian update, controlling how much the pulse shape is expected to change from pulse to pulse. This idea is called ‘Forward Phase-Lock Averaging,’ and the steps are given in “Algorithm 3” below.

FIG. 8 depicts both the von Mises-Fisher distribution and the pulse shape prior distribution, which can be derived by restricting the domain PDF of a multivariate Gaussian distribution. While they are similar distributions, the restriction that is used is different. The von Mises-Fisher distribution restricts the Gaussian to the unit sphere, while the pulse shape prior distribution restricts the Gaussian to its maximum value in a particular direction.

FIG. 9 compares the PDFs of the von Mises-Fisher distribution and Pulse Shape Prior distribution in terms of the angle θ. They are strikingly similar, except that the Pulse Shape Prior Distribution has the property that opposite angles share the same density. This is because opposite angles are considered the same direction for PLA, but for other applications in directional statistics that may not be desirable.

Stated generally, Algorithm 3, otherwise referred to herein as “Forward Phase-Lock Averaging,” comprises the following steps, which may, but need not always, be performed in the precise sequence listed below.

First, an initial estimate of the pulse Shape S0 may be made, in some cases along with a confidence factor φ.

Second, for k=1 to K the following steps may be iterated:

Estimate a prior amplitude under the assumption that Sₖ₋₁ is correct with the formula

$${α}_{k}^{prior} = \frac{{S}_{k - 1}^{T} {\sum }^{- 1} {S}_{k}^{detrend}}{{S}_{k - 1}^{T} {\sum }^{- 1} {S}_{k - 1}}$$

Create the matrix

$${S}^{stack} = [ \begin{matrix}\sqrt{φ} {α}_{k}^{prior} {S}_{k - 1} \\ \sqrt{1 - φ} {S}_{k}^{detrend}\end{matrix} ]$$

Compute the SVD of S^{stack}L and take the first singular value and vectors, σ₁, u₁, v₁.

As before, L may, in some cases, come from the Cholesky decomposition of Σ⁻¹.

Set

$${S}_{k} = \frac{{L}^{- 1} {v}_{1}}{{‖ {L}^{- 1} {v}_{1} ‖}_{2}} and {α}_{k} = {‖ {L}^{- 1} {v}_{1} ‖}_{2} {\partial }_{1} {u}_{1 , 2} .$$

In a similar way, we can incorporate prior information about the amplitude of the pulses. To do this, we assume a priori that the amplitudes and pulse shape are independent. Additionally, we assume a uniform prior distribution on the pulse shape and a Gaussian prior distribution on the amplitude with each aₖ˜N (aₖ^{prior}, σₐ). Doing this allows us to drop the pulse shape out of the loss function and only consider the amplitude. The loss function becomes:

$$\begin{matrix}f ( S , a ) = {‖ {S}^{detrend} L - {aS}^{T} L ‖}_{F}^{2} - 2 log P ( S , a ) \\ = {‖ {S}^{detrend} L - {aS}^{T} L ‖}_{F}^{2} - 2 log P ( S ) - 2 log P ( a ) \\ = \begin{matrix}{‖ {S}^{detrend} L - {aS}^{T} L ‖}_{F}^{2} - 2 log P ( a ) + {C}_{1} & ( Since P ( S ) is constant )\end{matrix} \\ = \begin{matrix}{‖ {S}^{detrend} L - {aS}^{T} L ‖}_{F}^{2} - {\sum }_{k = 1}^{K} \frac{{( {a}_{k}^{prior} - {a}_{k} )}^{2}}{{σ}_{a}} + {C}_{2} . & ( Absorbing constant terms )\end{matrix}\end{matrix}$$

Because constants do not contribute to the minimization, they can be ignored, so we will drop them out of the loss function. The next step is to rewrite the log PDF of a in a vector form so that it can neatly be brought into the summation:

$$\begin{matrix}f ( S , a ) = ( {\sum }_{k = 1}^{K} {( {S}_{k}^{detrend} - {a}_{k} S )}^{T} {\sum }^{- 1} ( {S}_{k}^{detrend} - {a}_{k} S ) ) + ( {\sum }_{k = 1}^{K} \frac{{( {a}_{k}^{prior} - {a}_{k} )}^{2}}{{σ}_{a}} ) \\ = {\sum }_{k = 1}^{K} {( {S}_{k}^{detrend} - {a}_{k} S )}^{T} {\sum }^{- 1} ( {S}_{k}^{detrend} - {a}_{k} S ) + \frac{{( {a}_{k}^{prior} - {a}_{k} )}^{2}}{{σ}_{a}} \\ = {\sum }_{k = 1}^{K} {( {S}_{k}^{detrend} - {a}_{k} S )}^{T} {\sum }^{- 1} ( {S}_{k}^{detrend} - {a}_{k} S ) + \frac{1}{T {σ}_{a}} ( {a}_{k}^{prior} - {a}_{k} ) {1}^{T} 1 ( {a}_{k}^{prior} - {a}_{k} ) \\ = {\sum }_{k = 1}^{K} {( {S}_{k}^{detrend} - {a}_{k} S )}^{T} {\sum }^{- 1} ( {S}_{k}^{detrend} - {a}_{k} S ) + \frac{1}{T {σ}_{a}} {( {a}_{k}^{prior} 1 - {a}_{k} 1 )}^{T} ( {a}_{k}^{prior} 1 - {a}_{k} 1 ) .\end{matrix}$$

Finally, by nothing that I=L⁻¹LL^{T}L^{-T}, we get that:

$$\begin{matrix}f ( S , a ) = {\sum }_{k = 1}^{K} {( {S}_{k}^{detrend} - {a}_{k} S )}^{T} {\sum }^{- 1} ( {S}_{k}^{detrend} - {a}_{k} S ) \\ + \frac{1}{T {σ}_{a}} {( {a}_{k}^{prior} 1 - {a}_{k} 1 )}^{T} {L}^{- 1} {LL}^{T} {L}^{- T} ( {a}_{k}^{prior} 1 - {a}_{k} 1 ) \\ = {\sum }_{k = 1}^{K} {( {S}_{k}^{detrend} - {a}_{k} S )}^{T} {\sum }^{- 1} ( {S}_{k}^{detrend} - {a}_{k} S ) \\ + \frac{1}{T {σ}_{a}} {( {a}_{k}^{prior} {L}^{- T} 1 - {a}_{k} {L}^{- T} 1 )}^{T} {\sum }^{- 1} ( {a}_{k}^{prior} {L}^{- T} 1 - {a}_{k} {L}^{- T} 1 ) \\ = {\sum }_{k = 1}^{K} {( {S}_{k}^{detrend} - {a}_{k} S )}^{T} {\sum }^{- 1} ( {S}_{k}^{detrend} - {a}_{k} S ) + {( {a}_{k}^{prior} w - {a}_{k} w )}^{T} {\sum }^{- 1} ( {a}_{k}^{prior} w - {a}_{k} w ) \\ = {\sum }_{k = 1}^{K} {( {S}_{k}^{detrend} + {a}_{k}^{prior} w - {a}_{k} ( S + w ) )}^{T} {\sum }^{- 1} ( {S}_{k}^{detrend} + {a}_{k}^{prior} w - {a}_{k} ( S + w ) ) \\ = {‖ ( {S}^{detrend} + {a}^{prior} {w}^{T} ) L - {a ( S + w )}^{T} L ‖}_{F}^{2}\end{matrix}$$

where we define:

$$w = \frac{1}{\sqrt{T {σ}_{a}}} {L}^{- T} 1 .$$

To solve this optimization problem, we can use the same method as was used in Theorem 3.1. However, there is an additional step that must be taken because computing the SVD of the matrix (S^{detrend}+a^{prior}w^{T})L and taking the first singular vectors and singular value only implies that â(Ŝ+w)^{T}L=u₁σ₁v^{T}₁. We must still solve for â and Ŝ. To do this, note that L^{T}(Ŝ+w)=φv₁ for some φ and that by constraint Ŝ^{T}Ŝ=1. Thus,

$${L}^{T} ( \hat{S} + w ) = φ {v}_{1}$$

$$\hat{S} + w = φ {L}^{- T} {v}_{1}$$

$$\hat{S} = φ {L}^{- T} {v}_{1} - w$$

$${\hat{S}}^{T} \hat{S} = {( φ {L}^{- T} {v}_{1} - w )}^{T} ( φ {L}^{- T} {v}_{1} - w )$$

$$1 = {φ}^{2} {v}_{1}^{T} {L}^{- 1} {L}^{- T} {v}_{1} - φ 2 {v}_{1}^{T} {L}^{- 1} w + {w}^{T} w$$

$$0 = {φ}^{2} {v}_{1}^{T} {L}^{- 1} {L}^{- T} {v}_{1} - φ 2 {v}_{1}^{T} {L}^{- 1} w + {w}^{T} w - 1.$$

Therefore, by the quadratic formula, we find that

$$φ = \frac{{v}_{1}^{T} {L}^{- 1} w \pm \sqrt{{( {v}_{1}^{T} {L}^{- 1} w )}^{2} - ( {v}_{1}^{T} {L}^{- 1} {L}^{- T} {v}_{1} ) ( {w}^{T} w - 1 )}}{{v}_{1}^{T} {L}^{- 1} {L}^{- T} {v}_{1}} .$$

This gives the optimal estimates for the pulse shape and amplitudes. Note that as before, we have a choice for the value of φ. Again, it is often best to choose the value of φ that makes â mostly non-negative; or, failing that, to choose the value of φ that favors the data over the prior.

$$\hat{S} = φ {L}^{- T} {v}_{1} - w$$

$$\hat{a} = \frac{{σ}_{1} {u}_{1}}{φ} .$$

A reasonable question to ask is whether q is always a real number. This is something that needs to be looked into more. Two limiting cases are easy: When σₐ→∞, then w→0, so the discriminant is

$$\sqrt{{v}_{1}^{T} {L}^{- 1} {L}^{- T} {v}_{1}} ,$$

which is clearly positive. This represents the case where no prior information is given about the amplitude. Likewise, as σₐ→0, w gets large, so L^{-T} v₁ approaches being colinear with w since Ŝ+w=φL^{-T} v₁. Then, by the Cauchy-Schwarz inequality in the special case of colinearity, we have that

$${( {v}_{1}^{T} {L}^{- 1} w )}^{2} = ( {v}_{1}^{T} {L}^{- 1} {L}^{- T} {v}_{1} ) ( {w}^{T} w ) .$$

Therefore, the discriminant is again

$$\sqrt{{v}_{1}^{T} {L}^{- 1} {L}^{- T} {v}_{1}} ,$$

which is positive. This represents the case where the amplitude is known exactly.

What happens in between these two limits is less clear. There is an argument to be made that it is possible for φ to be complex. By the Cauchy-Schwarz inequality, we have that:

$${( {v}_{1}^{T} {L}^{- 1} w )}^{2} \leq ( {v}_{1}^{T} {L}^{- 1} {L}^{- T} {v}_{1} ) ( {w}^{T} w )$$

$${( {v}_{1}^{T} {L}^{- 1} w )}^{2} - ( {v}_{1}^{T} {L}^{- 1} {L}^{- T} {v}_{1} ) ( {w}^{T} w ) \leq 0.$$

The only thing that is missing from the expression on the left-hand side is the v^{T}₁ L⁻¹L^{-T} v₁ term. This suggests that if

$$( {v}_{1}^{T} {L}^{- 1} {L}^{- T} {v}_{1} ) ( {w}^{T} w ) - {( {v}_{1}^{T} {L}^{- 1} w )}^{2} \geq {v}_{1}^{T} {L}^{- 1} {L}^{- T} {v}_{1} ,$$

then φ is complex. This is a difficult inequality to solve, and it is not clear under what circumstances it is true because v₁ comes from the SVD of a matrix that is dependent on the data and the prior. However, an observation that can be made is that w is unbounded as σₐ→0 while v₁ is fixed to have norm 1, so it is likely that there is some region of σₐ where φ is complex. But, as shown above, there must be a point where φ becomes real again as σₐ approaches 0, since in the limiting case q is real and expressions involved are continuous in σₐ.

The region where φ is complex (if it exists) can be interpreted as a region in which the prior information is incompatible with the data.

It is difficult to evaluate the effectiveness of any filter on real data, because the true signal is not known. Indeed, filters are applied to try to find the true signal. Phase-Lock Averaging is no different.

Because of this, all that we can do is evaluate the effectiveness of PLA on synthetically generated data, and use insights from the synthetic data to glean information about how well PLA is expected to perform on real data. This will be addressed below. Thereafter, a discussion about how well PLA addresses the problems with Fourier methods that were brought up previously will ensue.

It was shown throughout this disclosure that PLA is the optimal estimator under the assumptions made, as presented above. However, it is also worth considering how well PLA actually performs under these assumptions (the most important assumption being white noise). In addition, it is worth exploring how well PLA performs when the data deviates somewhat from the assumptions of the model. Specifically, because real-world PPG data contains non-trivial amounts of pink noise, it may be useful to see how well PLA performs in the presence of pink noise. Therefore, in addition to the white noise case, the examples below demonstrate how PLA performs under pure pink noise, and a combination of 50% white noise and 50% pink noise (most similar to what an actual PPG device would measure).

Finally, because the assumption was made that the trend of the signal has been removed and a procedure has been given to remove the trend, it may also be useful to see how the detrending procedure affects the PLA estimates.

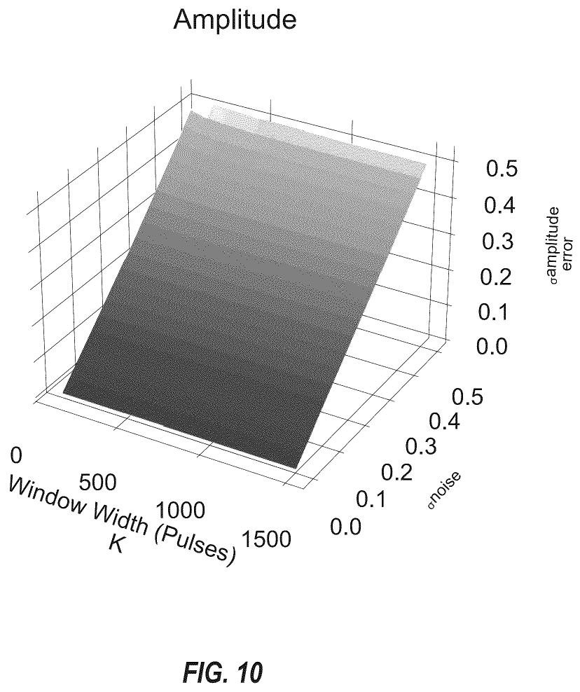

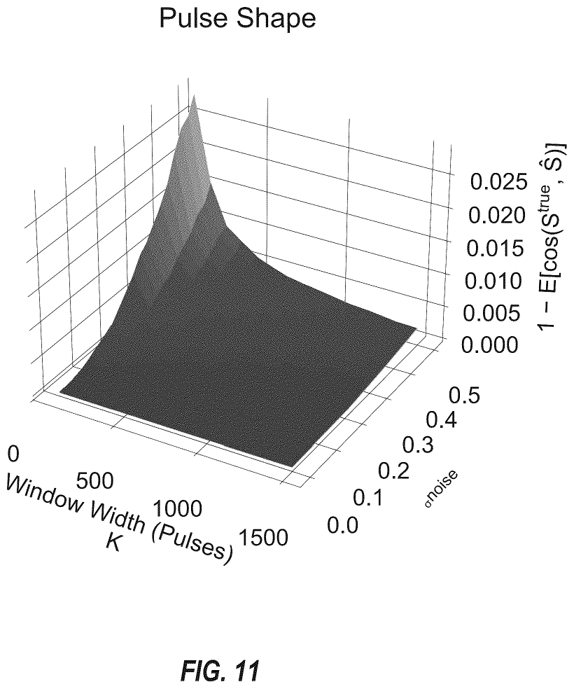

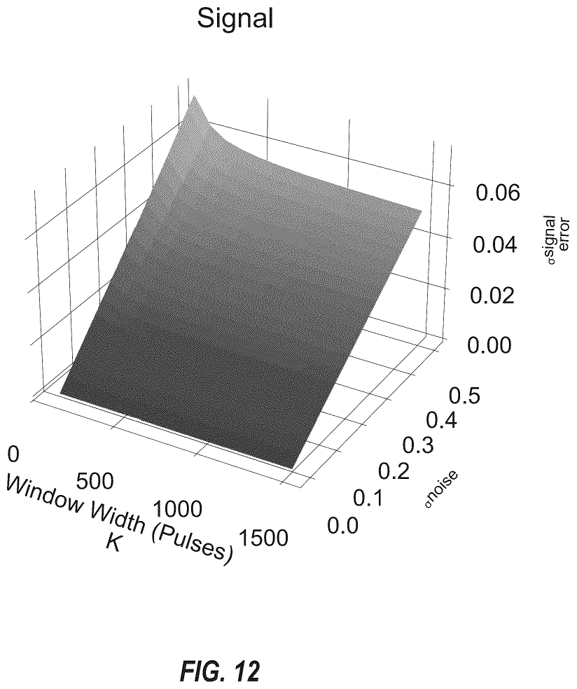

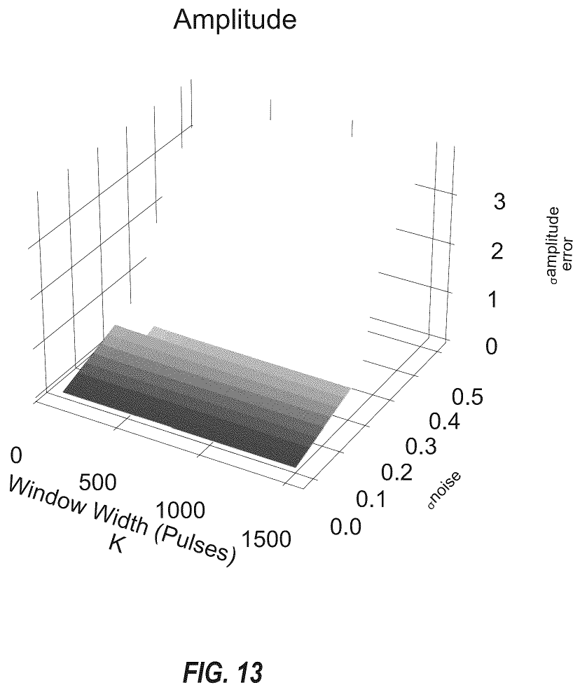

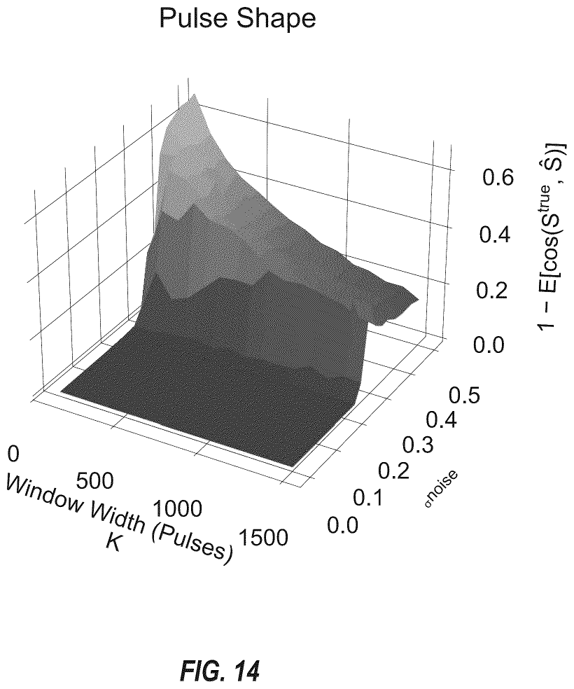

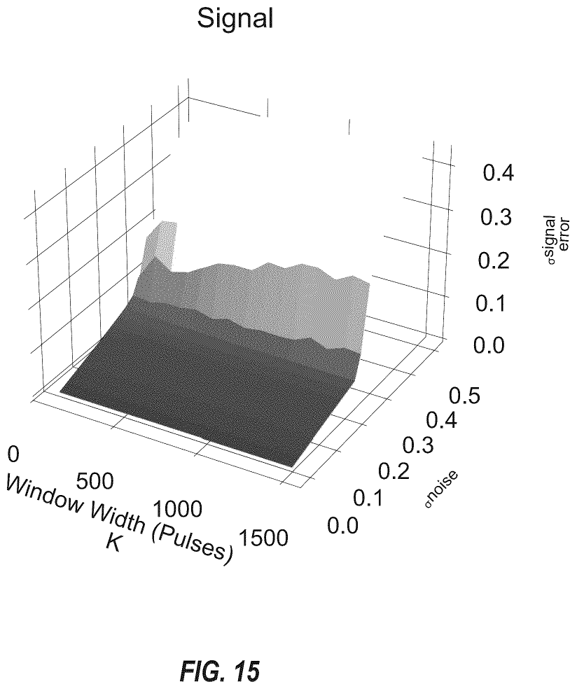

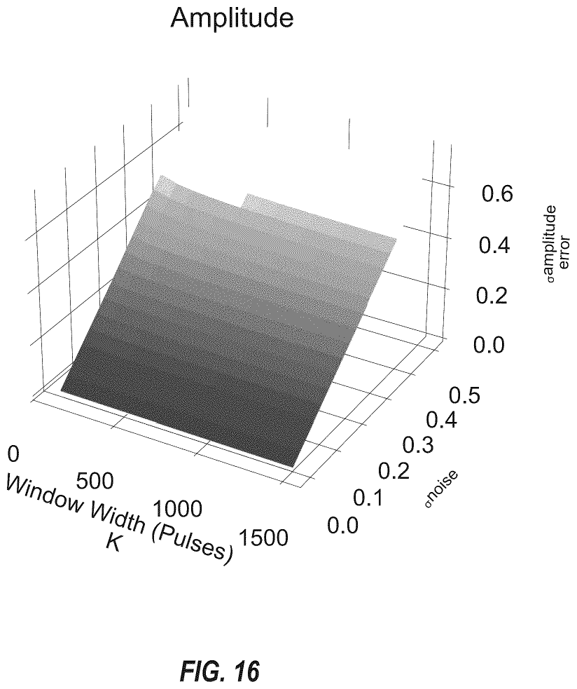

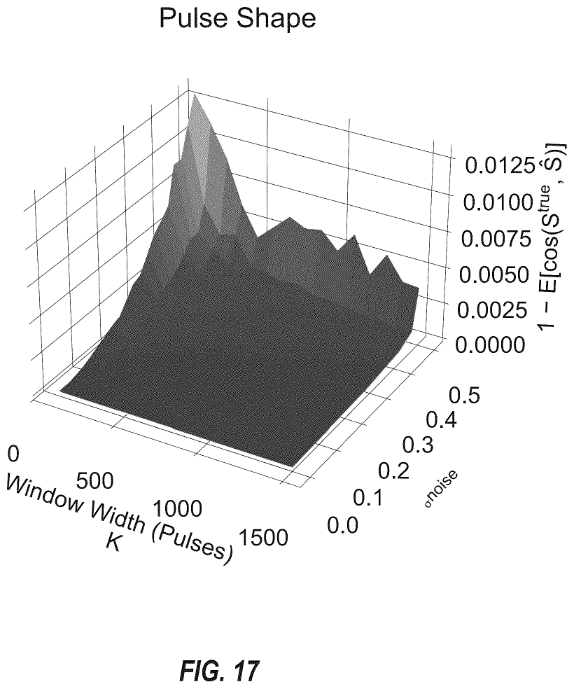

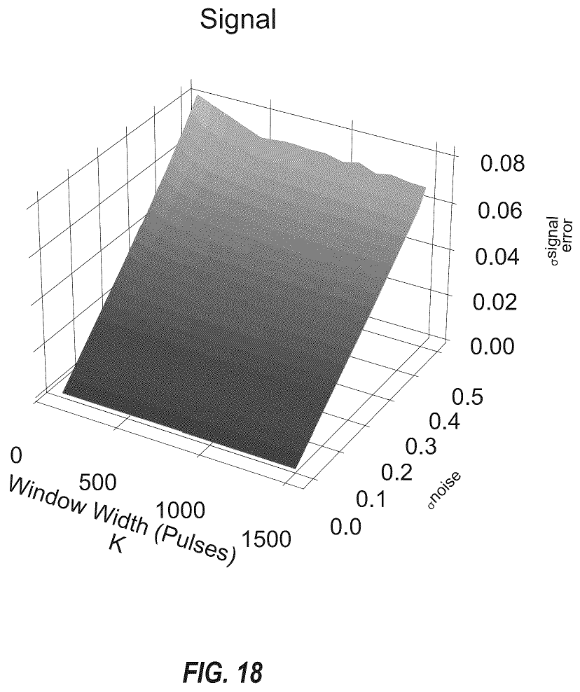

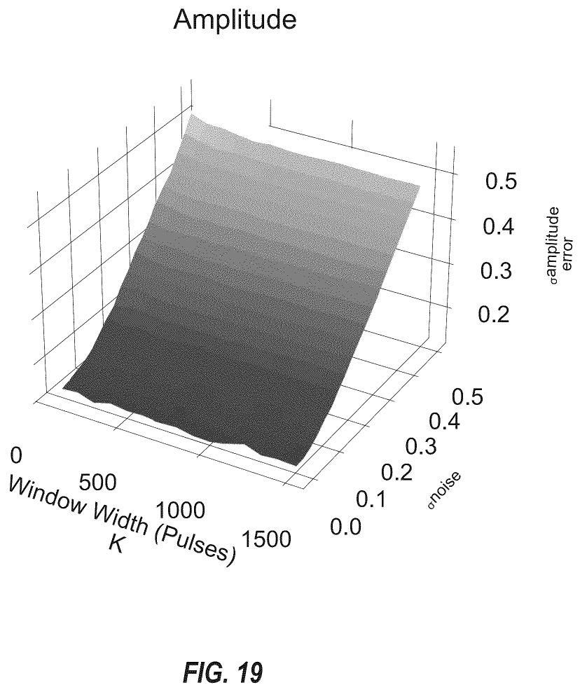

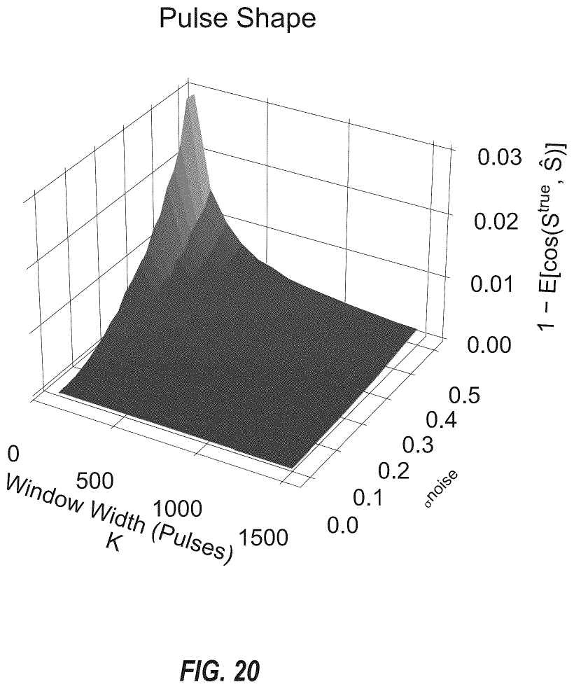

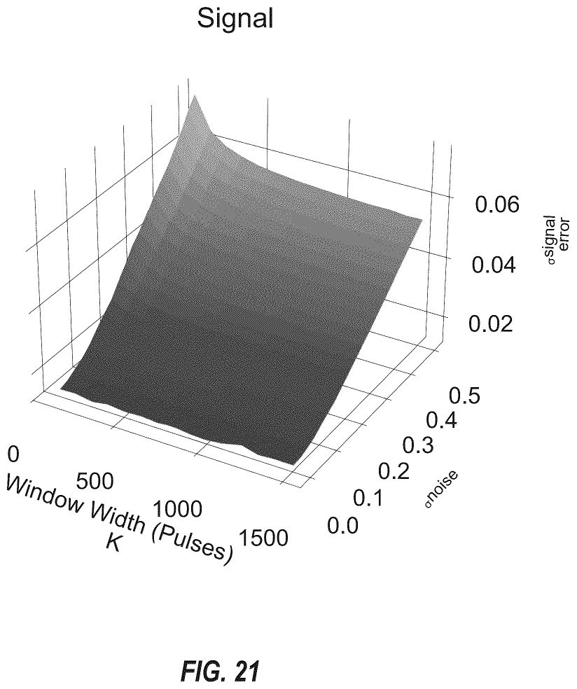

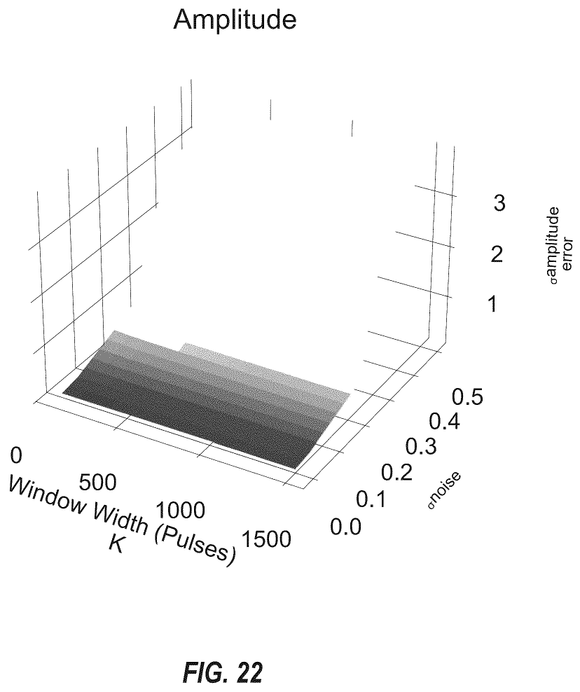

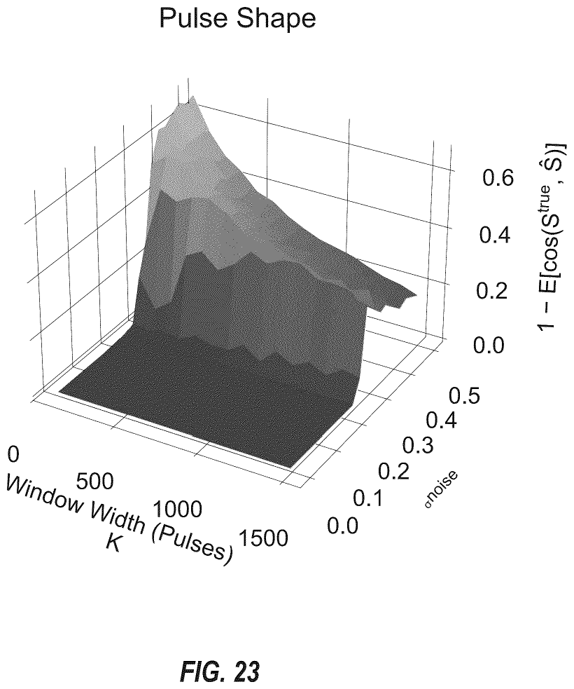

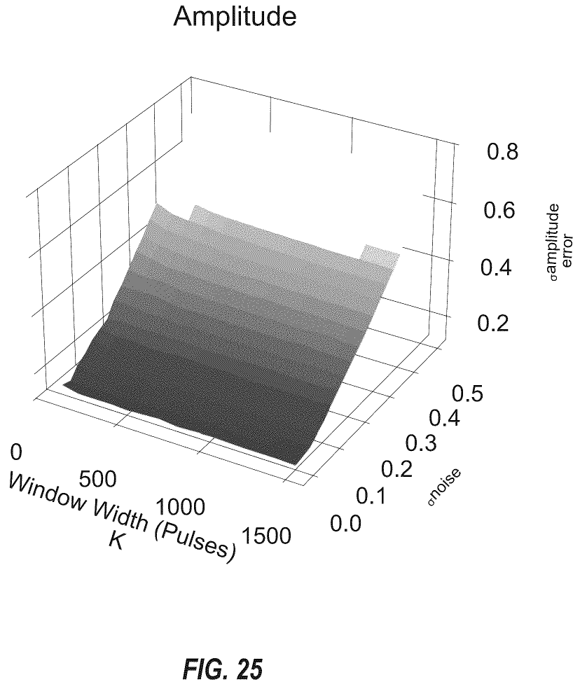

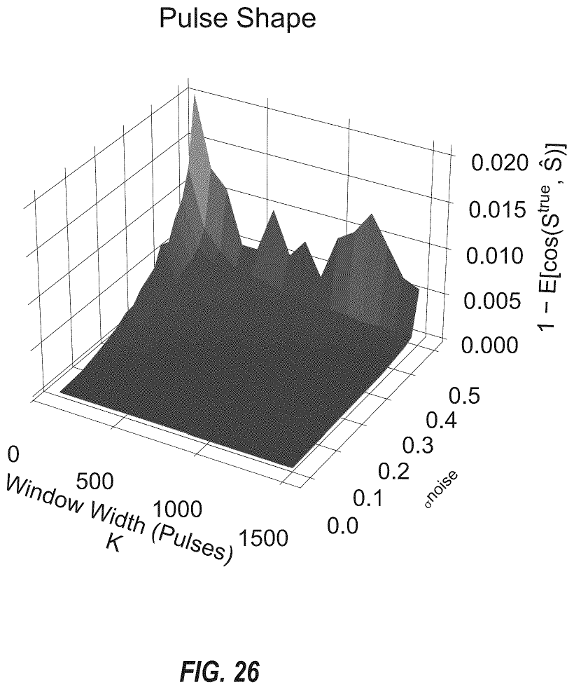

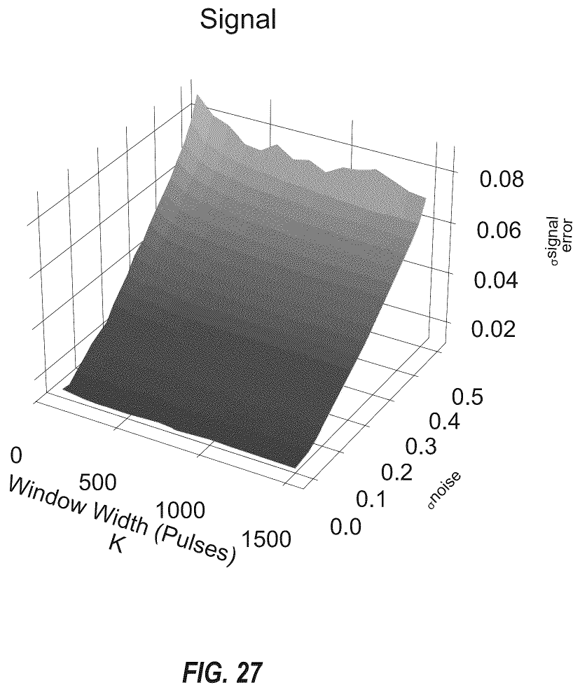

To test the performance of PLA, we performed a large monte carlo experiment, six conditions were tested:
(i) White noise with no detrending (See FIGS. 10-12);
(ii) Pink noise with no detrending (See FIGS. 13-15);
(iii) Combination of 50% white and 50% pink noise with no detrending (See FIGS. 16-18);
(iv) White noise with detrending (See FIGS. 19-21);
(v) Pink noise with detrending (See FIGS. 22-24); and
(v₁) Combination of 50% white and 50% pink noise with detrending (See FIGS. 25-27).

For each condition, the following steps were performed:
(i) Create a synthetic pulse (S^{true}) of length 100, which is 1 second long if the PPG device measures at a rate of 100 Hz;
(ii) For every choice of window width K=100, 200, . . . , 1500 and for every choice of standard deviation for the noise o^{noise}=0.0, . . . , 0.5, create 10,000 synthetic signals:
(a) Draw K random amplitudes from N (2, 0.5);
(b) Create the signal by repeating the pulse shape K times and multiplying it by the K random amplitudes. This gives a signal with a power of approximately 0.043.
(c) Draw 100K values from the noise distribution (white, pink, or combination) with a standard deviation of σ^{noise}. This gives noise with a power of σ^{noise2};
(d) Add the signal to the noise;
(e) Apply detrending to the signal if required;
(f) Perform PLA (Algorithm 1) to get the pulse shape estimate and amplitude estimates;
(g) Construct a signal estimate from the estimated pulse shape and amplitudes;
(h) Compute residuals for amplitude estimates and signal estimates;
(i) Compute the cosine distance for the pulse shape estimate (1−cos(S^{true}, Ŝ));
(iii) Using the 10,000 samples from each window width and choice of standard deviation, compute the RMSE of the estimated amplitudes

$$( {σ}_{error}^{amplitude} )$$

and the estimated signal

$$( {σ}_{error}^{signal} )$$

using the residuals. Compute the expected value of the cosine distances of the pulse shapes; and
(iv) Store these three values and plot them.

The results of this giant monte carlo experiment can be seen in FIGS. 10-27.

In FIGS. 10-12, Monte carlo results for PLA applied to a signal with varying amounts of white noise added are shown. PLA was applied directly without first detrending the signal. The upper left region in the amplitude plot in FIG. 10 represents the point at which the same amount of error or better can be achieved by instead estimating all amplitudes as the mean of the amplitudes. Notice that there appears to be a perfectly linear relationship between the amplitude error and the standard deviation of the noise. Increasing the window width only improves the amplitude estimates slightly, but significantly improves the pulse shape estimates. Overall, as the window width increases, there begin to be diminishing returns in the error of the signal estimate. This figure represents the best-case scenario and is a comparison point for the later figures.

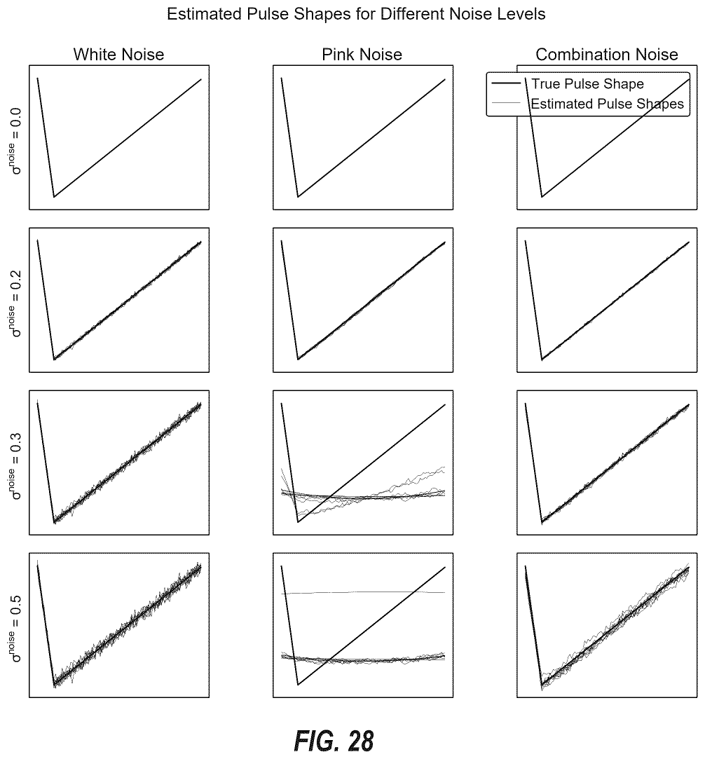

FIGS. 13-15 illustrate monte carlo results for PLA applied to a signal with varying amounts of pink noise added. PLA was applied directly without first detrending the signal. The white region in the amplitude plot represents the point at which the same amount of error or better can be achieved by instead estimating all amplitudes as the mean of the amplitudes. The white region in the signal plot represents the point at which the same amount of error or better can be achieved by instead estimating the signal as its mean (i.e., no signal). This figure shows that pink noise is severely detrimental to the performance of PLA. The most prominent feature of this figure is the pulse shape plot, where it can be seen that there is a steep cliff followed by a flat shelf as the amount of noise increases. The point where the cliff appears is approximately the point where the signal-to-noise ratio is 1:2. Beyond this point, PLA is unable to accurately estimate the pulse shape, which detrimentally affects the amplitude estimates, and consequently the signal estimate. Compare this to FIGS. 10-12. See also FIG. 28 for an explanation as to why the cliff appears.

FIGS. 16-18 depict monte carlo results for PLA applied to a signal with varying amounts of a combination of 50% white noise and 50% pink noise added. PLA was applied directly without first detrending the signal. The white region in the amplitude plot of FIG. 16 represents the point at which the same amount of error or better can be achieved by instead estimating all amplitudes as the mean of the amplitudes. Notice that even though FIGS. 13-15 show that pink noise is detrimental to the performance of PLA, having a combination of white noise and pink noise is only slightly worse than having plain white noise. That is, until right around the point where σ^{noise}=0.5, where we notice the beginnings of a cliff starting to form in the pulse shape plot. The signal-to-noise ratio at this point is approximately 1:4, meaning that the ratio of signal to pink noise is about 1:2. Finally, another important thing to note is that even though there is a white region in the amplitude plot, the signal plot still shows that the estimate in the signal is good.

FIGS. 19-21 depict monte carlo results for PLA applied to a signal with varying amounts of white noise added. To see how detrending the signal affects the PLA estimates, detrending was performed before PLA. The white region in the amplitude plot of FIG. 19 represents the point at which the same amount of error or better can be achieved by instead estimating all amplitudes as the mean of the amplitudes. These figures are very similar to FIGS. 10-12. However, in those figures, as the amount of noise goes to 0, the error in the amplitude estimation and signal estimation also goes to 0. Here, there is a small amount of error that was introduced by the detrending. Notice, though, that once there is enough noise, the error introduced by the detrending seems to almost entirely disappear, and the plots look nearly identical to those in FIGS. 10-12. Pulse shape, too, is affected, but only slightly and only when the window width is small.

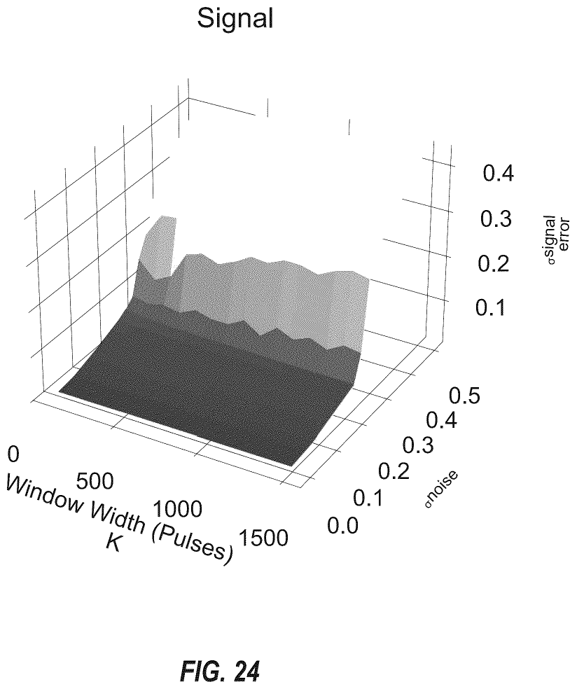

FIGS. 22-24 depict monte carlo results for PLA applied to a signal with varying amounts of pink noise added. To see how detrending the signal affects the PLA estimates, detrending was performed before PLA. The white region in the amplitude plot of FIG. 22 represents the point at which the same amount of error or better can be achieved by instead estimating all amplitudes as the mean of the amplitudes. The white region in the signal plot of FIG. 24 represents the point at which the same amount of error or better can be achieved by instead estimating the signal as its mean (i.e., no signal). Because pink noise ‘wanders around’ a bit, it was hoped that detrending the signal before PLA would make pink noise behave more like white noise. As can be seen in this figure, though, detrending does nothing to ease the effects of pink noise on PLA. These plots appear to be nearly identical to those of FIGS. 13-15.

FIGS. 22-24 depict monte carlo results for PLA applied to a signal with varying amounts of a combination of 50% white noise and 50% pink noise added. To see how detrending the signal affects the PLA estimates, detrending was performed before PLA. The white region in the amplitude plot of FIG. 22 represents the point at which the same amount of error or better can be achieved by instead estimating all amplitudes as the mean of the amplitudes. The signal plot of FIG. 24 represents something similar to the expected conditions that would surround a typical PPG signal. It is a mix of white and pink noise, and because the baseline of the signal gradually changes over time in a typical PPG signal, detrending would be necessary before PLA. PLA does fairly well here, despite deviations from its assumptions. The same observations made in FIGS. 16-21 can be seen here.

One might wonder why the expected cosine distance was chosen as an error metric for the pulse shape estimates. The reason for this is that because the pulse shape estimates are restricted to the unit sphere in R¹⁰⁰, (really, they are restricted to the unit half sphere, since PLA will arbitrarily choose one of the vectors v and −v for all v∈R¹⁰⁰) it doesn't make sense to simply compute the MSE between the true pulse shape and the estimated pulse shape. However, the expected cosine distance is a similar metric. Consider that:

$$\begin{matrix}MSE ( {S}^{true} , \hat{S} ) = 𝔼 [ \frac{1}{100} {\sum }_{t = 1}^{100} {( {S}^{true} , {\hat{S}}_{t} )}^{2} ] \\ = \frac{1}{100} 𝔼 [ {‖ {S}^{true} - \hat{S} ‖}_{2}^{2} ] \\ = \frac{1}{100} 𝔼 [ {S}^{{true}^{T}} {S}^{true} - 2 {S}^{{true}^{T}} \hat{S} + {\hat{S}}^{T} \hat{S} ] \\ = \frac{1}{100} 𝔼 [ 2 - 2 {S}^{{true}^{T}} \hat{S} ] \\ = \frac{1}{50} ( 1 - 𝔼 [ cos ( {S}^{true} , \hat{S} ) ] ) .\end{matrix}$$

Thus, the expected cosine distance behaves like a scaled version of the MSE.

The difficulty with this metric, though, is that it is not as easy to interpret as the RMSE. In general, humans have bad intuition about high dimensions. Because of that, it is difficult to tell what a good score to have is and what a bad score to have is. To make it easier to see, FIG. 28 shows the distribution of pulse shapes for a few values of σ^{noise} when the window width is restricted to 600 (looking at the pulse shape plots of FIGS. 10-24, 600 the point where there start to be diminishing returns for increasing the window width).

Some key takeaways from the monte carlo experiment are that:
(i) PLA performs very well even at high levels of noise (still performs well with a signal-to-noise ratio of 1:5);
(ii) Pink noise is detrimental to the performance of PLA, but PLA still performs well until the ratio of signal to pink noise is about 1:2. It is unclear exactly why a ratio of 1:2 is the point where a cliff appears, but at this point, PLA begins to simply estimate a flat pulse shape, which drastically affects the amplitude estimates and consequently the signal estimate;
(iii) Amplitude and signal estimate errors increase approximately linearly with increasing noise standard deviation;
(iv) Amplitude estimate error is barely affected by increasing the window width;
(v) Pulse shape estimates get better with increasing window width, but there start to be diminishing returns beyond around 600 pulses; and
(v₁) Detrending the signal introduces a small bias into the estimates, but as the noise increases, the effect of the bias becomes negligible. Therefore, detrending appears to do no harm. It also does not appear to help to remove the ‘wander’ of pink noise.

We will now consider whether PLA solves the problems that Fourier Methods have, as discussed above. The answer is yes.

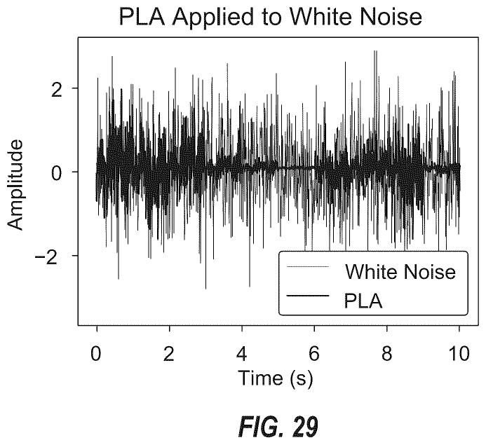

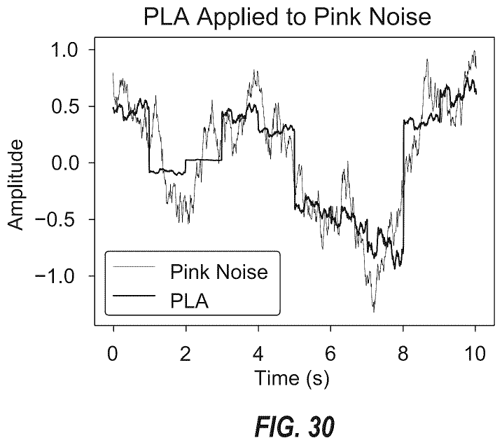

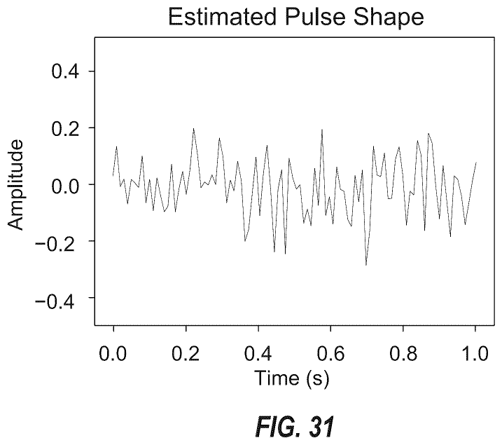

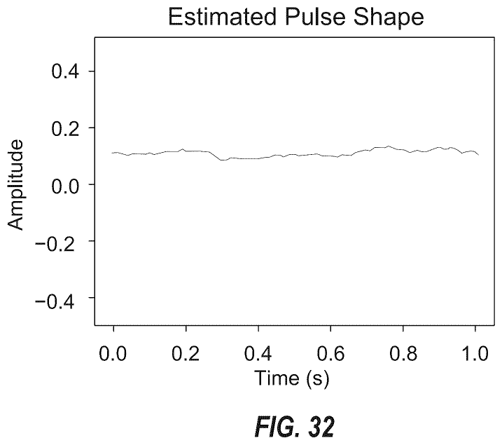

One of the main issues with Fourier methods is that they pass noise through, but it does not look like noise. This happens because the filter smooths out the noise a bit by only allowing its low-frequency parts through. PLA does not have this issue because the noise that makes it through PLA shows as noise in the pulse shape estimate or noise in the amplitude estimates. As seen in FIGS. 29-32, when PLA is applied to pure noise, the estimates that come out the other side still look noisy, so there is less possibility of thinking that there is a signal where there is none.

FIGS. 29-32 show PLA applied to pure noise. Compare this to FIGS. 4A and 4B. Notice that, in both cases, while PLA does produce an estimate for pulse shape and amplitudes, the reconstructed signal still looks like noise, unlike Fourier methods. Interesting to note is that while the pulse shape estimate for white noise is an erratic, noisy shape, the pulse shape estimate for pink noise is a bumpy line. This is likely because of the wandering nature of the pink noise. PLA effectively finds a step function approximation to the pink noise, then puts some random bumps in to minimize the error. In the pink noise plot, even though the pulse shape estimate looks like a line, the signal estimate is somewhat bumpy because the small bumps are magnified by the amplitudes as the pulse shape is multiplied to create the signal.

Artifacts in the cleaned signal from Fourier methods are not always due to noise. If the filter is not designed well, then the filter will not be able to accurately reproduce the pulse shape. PLA is especially focused on being able to recover the pulse shape accurately without introducing artifacts. It does this job well. However, one obvious issue with PLA that we have entirely ignored until this point is in how PLA reconstructs a cleaned signal.

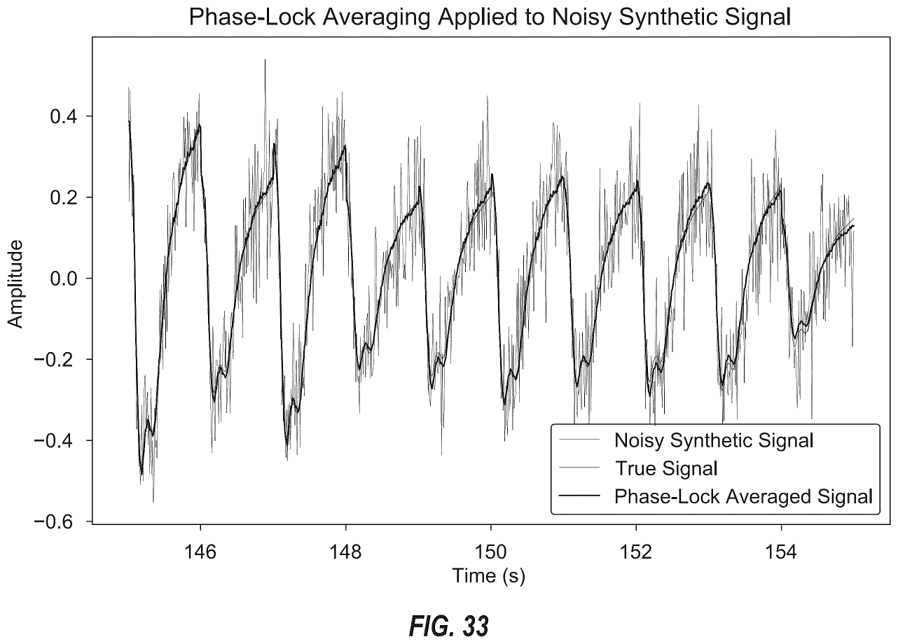

Because all that it does is glue a bunch of pulse shapes together, multiplied by their predicted amplitudes, PLA can have sharp transitions from pulse to pulse. This can be seen in FIG. 33. Though PLA recovers the true signal better than Fourier methods do, the sharp corners between pulses are not something that is observed in real-world PPG signals. These artifacts are clearly undesirable, but they are the price that is paid for being able to recover pulse shape much more accurately than any Fourier method can.

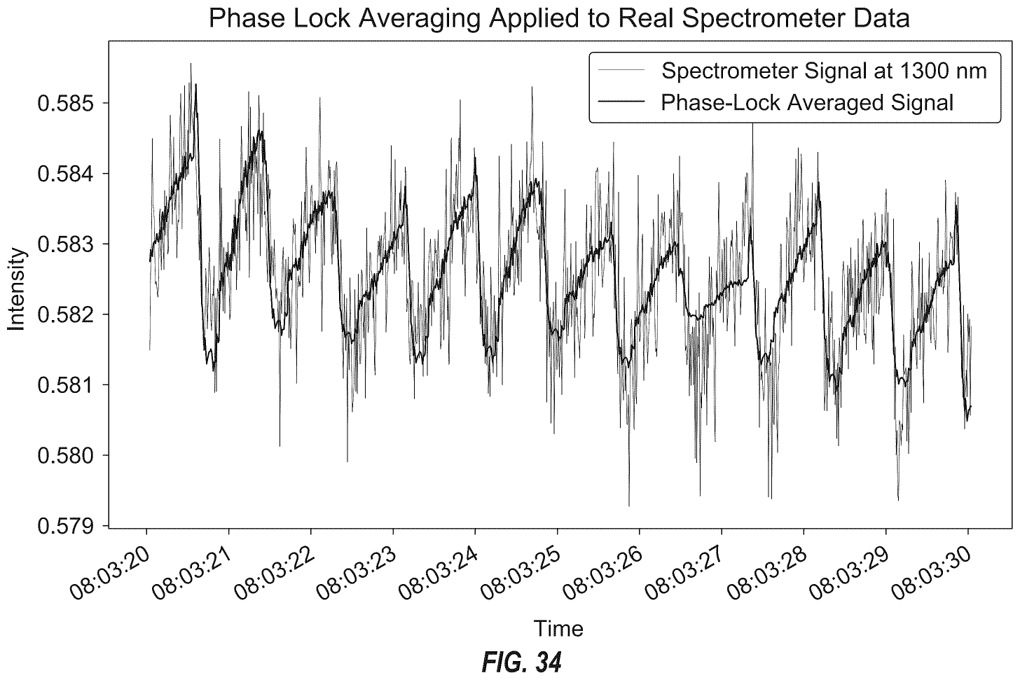

In FIG. 34, we see PLA applied to real spectrometer data to see an example of how well it performs in practice.

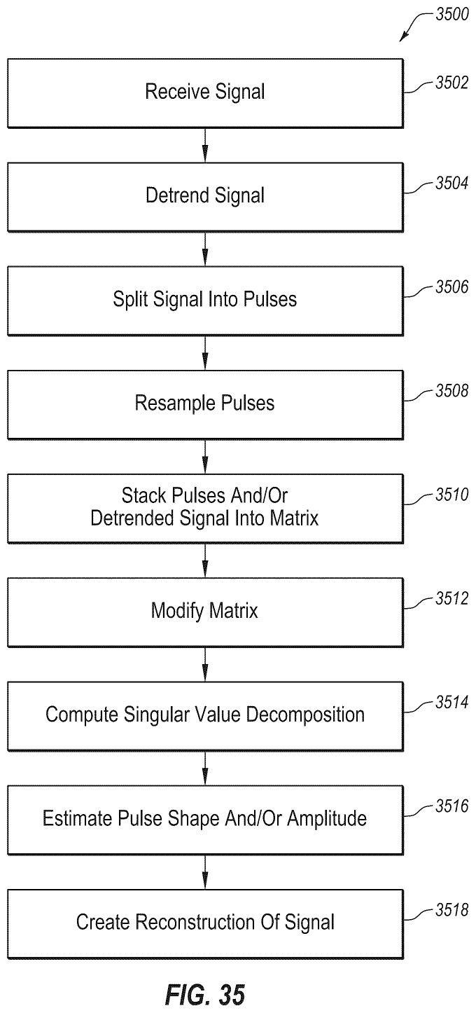

FIG. 35 is a flowchart of a method 3500 according to some implementations. At step 3502, a signal is received. In some cases, the signal may comprise a physiological signal, such as a heartbeat signal. The signal may be received, for example, by a non-invasive blood monitor comprising one or more sensors, which may be incorporated into a wristband to allow for detection of reflected signals from a user's radial artery. Examples of such monitors can be found in U.S. Pat. No. 11,903,686 titled “Systems, Apparatuses, and Methods for Optimizing a Physiological Measurement Taken From a Subject,” the entire contents of which are hereby incorporated herein by reference in its entirety.

After receiving the signal, the signal may be detrended at 3504. The signal may then be split at 3506. In some cases, the signal may be split into pulses and/or vectors. In some cases, a peak-finder may be used to split the signal, such as for heartbeat signals into individual heartbeats/pulses.

The pulses may then be resampled at 3508. For example, in some cases, each pulse may be resampled to a common length.

The pulses and/or detrended signal may then be stacked into one or more matrices at step 3510. In some cases, the one or more matrices may be modified at 3512, preferably in a manner that facilitates recovery after taking a singular value decomposition. For example, in some cases, the matrix/matrices may be modified by means of matrix multiplication, weighting, or mathematical decomposition. In some preferred implementations, this modification may comprise multiplying the stacked matrix or stacked matrices by the Cholesky decomposition of the inverse covariance matrix. In other cases, the stacked matrix or stacked matrices may be multiplied by a weighting matrix. As still another possibility, in some cases, an Eigen decomposition of the inverse covariance matrix may be used.

The Singular Value Decomposition (SVD) may then be computed at step 3514. The results of the SVD may allow for estimating the shape(s) and/or amplitude(s) of the pulse(s) at step 3516, as discussed throughout this disclosure.

The signal, or a portion of the signal, may then be reconstructed at step 3518. In some cases, the trend, pulse shape(s), and/or amplitude(s) may be combined to create this reconstructed signal.

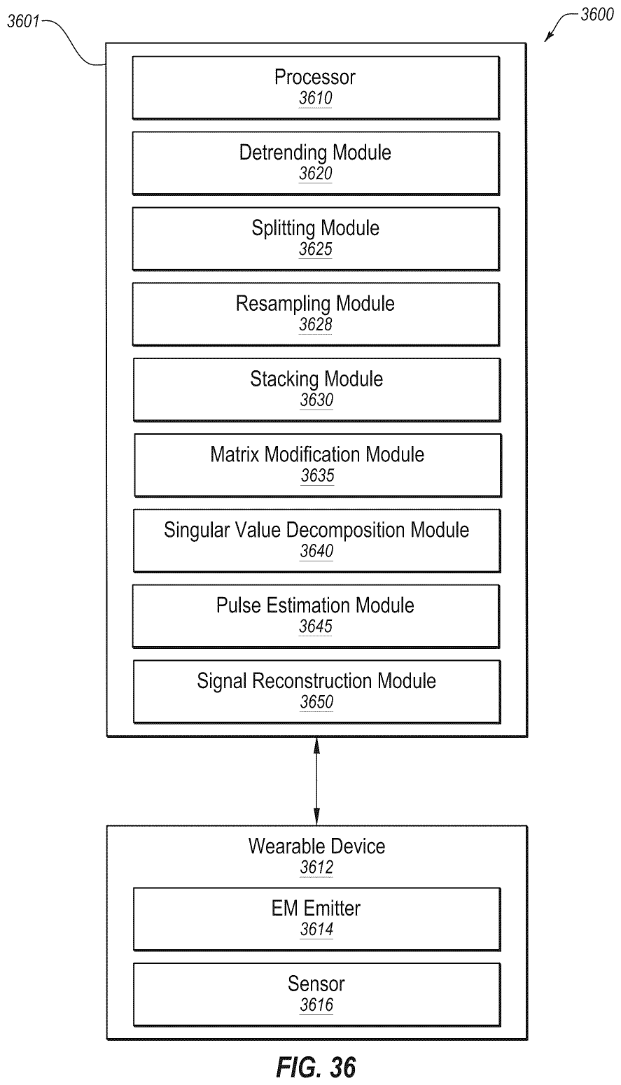

FIG. 36 is a schematic diagram of a system 3600 for processing data, such as data from a wearable device 3612, which may be used for monitoring health conditions, such as blood analyte conditions, according to some implementations and embodiments. As shown in this figure, wearable device 3612, which may comprise, for example, a wrist monitor, comprises one or more electromagnetic emitters 3614 and one or more sensors 3616. In some embodiments, the wearable device 3612 may be positioned on the wrist so that a physiological sensor 3616 is positioned adjacent to a particular, targeted physiological feature, such as an ulnar artery or radial artery, for example.

In some embodiments, one or more electromagnetic emitters 3614 may be positioned at a suitable location relative to the sensor(s) 3616 so that electromagnetic radiation of a desired wavelength may be emitted from emitter(s) 3614, reflected from desired regions within the body, such as along an adjacent to a particular artery in the case of blood monitoring, and then received by sensor(s) 3616 for processing according to, for example, the method of FIG. 35 or any of the other methods disclosed herein.

As those of ordinary skill in the art will appreciate, sensor(s) 3616 may be configured to convert reflected/incoming radiation into electronic signals, which may be transmitted to and received by processing system 3601. For example, in some embodiments, sensor 3616 may be configured to measure the intensity of the received radiation over time and transmit this signal to system 3601. System 3601 may be incorporated onto the wearable device 3612 or may be incorporated into another device, such as a mobile application running on a smartphone, a computer, a remote server, or the like.

System 3601 may comprise one or more processors 3610. Processor(s) 3610 may be configured to receive and/or process data from wearable device 3612 and/or sensor(s) 3616. In addition, processor(s) 3610 may be configured to perform various functions, some of which may be part of a particular software module. As used herein, a software module or component may include any type of computer instruction or computer executable code located within a memory device and/or m-readable storage medium. A software module may, for instance, comprise one or more physical or logical blocks of computer instructions, which may be organized as a routine, program, object, component, data structure, etc., that perform one or more tasks or implements particular abstract data types.

System 3601 may comprise a Detrending Module 3620, which may be operably coupled with processor 3610 and/or one or more of the other modules of the system 3601. Detrending Module 3620 may be configured to estimate a trend in one or more portions of a signal and/or remove the trend from the signal(s). Various methods for estimating and/or detrending are discussed throughout this disclosure, any one of which may be performed by the Detrending Module.

System 3601 may further comprise a Splitting Module 3625, which may be operably coupled with processor 3610 and/or one or more of the other modules of the system 3601. Splitting Module 3625 may be configured to split one or more signals into individual pulses, such as individual heartbeats. In some cases, a peak-finder may be used to accomplish this splitting.

System 3601 may further comprise a Resampling Module 3628, which may be operably coupled with processor 3610 and/or one or more of the other modules of the system 3601. Resampling Module 3628 may be configured to resample one or more portions of the signal(s). For example, in some cases, individual heartbeats may be resampled, such as resampled to be the same length.

System 3601 may further comprise a Stacking Module 3630, which may be operably coupled with processor 3610 and/or one or more of the other modules of the system 3601. Stacking Module 3630 may be configured to stack pulses obtained from the signal, such as heartbeats, into one or more matrices, as discussed above in greater detail. In some cases, Stacking Module 3630 may be configured to stack resampled heartbeats from a physiological signal into the matrix/matrices.

System 3601 may further comprise a Matrix Modification Module 3635, which may be operably coupled with processor 3610 and/or one or more of the other modules of the system 3601. Matrix Modification Module 3635 may be configured to modify the stacked matrix by means of matrix multiplication, weighting, and/or mathematical decomposition. For example, in some cases Matrix Modification Module 3635 may be configured to multiply the matrix by the Cholesky decomposition of the inverse covariance matrix. In some cases, the Eigen decomposition of the inverse covariance matrix may be used.

System 3601 may further comprise a Singular Value Decomposition Module 3640, which may be operably coupled with processor 3610 and/or one or more of the other modules of the system 3601. Singular Value Decomposition Module 3640 may be configured to compute the singular value decomposition of one or more of the matrices, such as matrixes stacked using Stacking Module 3630. Various techniques for taking the Singular Value Decomposition, along with related tasks, any of which may also be performed by Singular Value Decomposition Module 3640, are discussed throughout this disclosure, such as weighting the pulses, scaling, etc.

System 3601 may further comprise a Pulse Estimation Module 3645, which may be operably coupled with processor 3610 and/or one or more of the other modules of the system 3601. In some embodiments, the shape of one or more pulses may be estimated using Pulse Estimation Module 3645. In some embodiments, the amplitude(s) of one or more pulses may also, or alternatively, be estimated using Pulse Estimation Module 3645. Techniques for taking such estimates are discussed throughout this disclosure.

System 3601 may further comprise a Signal Reconstruction Module 3650, which may be operably coupled with processor 3610 and/or one or more of the other modules of the system. Signal Reconstruction Module 3650 may be configured to combine one or more estimates from Pulse Estimation Module 3645 and create a reconstruction of the signal. Techniques for taking such reconstructions are discussed throughout this disclosure.

In certain embodiments, a particular software module may comprise disparate instructions stored in various locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. Examples of suitable modules that may be used include MATLAB, Python with Numpy, and LAPACK.

In addition, data being tied or rendered together in a database record may be resident in the same memory device, or across several memory devices, and may be linked together in fields of a record in a database across a network. Furthermore, embodiments and implementations of the inventions disclosed herein may include various steps, which may be embodied in machine-executable instructions to be executed by a general-purpose or special-purpose computer (or another electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMs, EPROMs, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions s configured for execution by a processor, controller/control unit, or the like.

It will be understood by those having ordinary skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. The scope of the present invention should, therefore, be determined only by the following claims.

1. A method for reducing signal noise from a non-invasive blood analyte monitoring system, the method comprising the steps of:
receiving physiological signal data from a non-invasive blood monitor;
estimating a trend in the physiological signal data;
detrending at least a portion of the physiological signal data to create a detrended signal;
splitting the detrended signal into vectors;
stacking the detrended signal into a matrix; and
obtaining an estimate for a shape and/or amplitude of a pulse associated with the physiological signal.

2. The method of claim 1, further comprising taking a decomposition of the matrix.

3. The method of claim 2, further comprising modifying a stacked matrix using matrix multiplication, weighting, and/or mathematical decomposition.

4. The method of claim 2, wherein the decomposition comprises a Cholesky decomposition.

5. The method of claim 1, wherein the step of obtaining an estimate for a shape and/or amplitude of a pulse associated with the physiological signal comprises using a singular value decomposition of the matrix to estimate the shape and/or amplitude of the pulse.

6. The method of claim 1, wherein the non-invasive blood monitor is configured to detect a concentration of a blood analyte.

7. The method of claim 6, wherein the blood analyte comprises glucose.

8. A method for removing noise from signal data obtained from a non-invasive blood monitor, the method comprising the steps of:
receiving signal data from a non-invasive blood monitor;
detrending at least a portion of the signal data to create a detrended signal;
stacking the detrended signal into a matrix; and
taking a singular value decomposition of the matrix.

9. The method of claim 8, further comprising taking a Cholesky decomposition of an inverse covariance matrix derived from the matrix.

10. The method of claim 8, further comprising using the singular value decomposition to estimate a shape and/or amplitude of a pulse associated with the signal data.

11. The method of claim 10, wherein the step of using the singular value decomposition to estimate a shape and/or amplitude of a pulse associated with the signal data comprises estimating the shape and the amplitude of the pulse.

12. The method of claim 8, wherein the signal data comprises physiological signal data.

13. The method of claim 12, further comprising estimating a concentration of a blood analyte using the physiological signal data.

14. The method of claim 13, wherein the blood analyte comprises glucose.

15. A system for removing noise from signal data, comprising:
a radiation generator configured to generate electromagnetic radiation;
a sensor configured to receive reflected electromagnetic radiation from the radiation generator;
a detrending module configured to detrend a signal from data obtained from the sensor;
a stacking module configured to stack a detrended signal obtained from the detrending module;
a singular value decomposition module configured to take a singular value decomposition of the detrended signal; and
a signal reconstruction module configured to obtain a pulse shape and/or a one or more pulse amplitudes from the singular value decomposition.

16. The system of claim 15, wherein the signal data comprises physiological signal data.

17. The system of claim 16, wherein the physiological signal comprises a heartbeat.

18. The system of claim 15, further comprising a triangular decomposition module configured to compute a decomposition of an inverse covariance matrix derived from the signal data.

19. The system of claim 18, wherein the triangular decomposition module comprises a Cholesky decomposition module.

20. The system of claim 15, wherein the sensor comprises a spectrometer, and wherein the signal comprises a photoplethysmography signal.
